(19) United States
(12) Patent Application Publication
(10) Pub. No.: US 2009/0163561 A1
(43) Pub. Date: Jun. 25, 2009

(54) AMORPHOUS METAXALONE AND AMORPHOUS DISPERSIONS THEREOF

(75) Inventors: Keith Lorimer, West Lafayette, IN (US); Kevin Wayne Meyer, Lebanon, IN (US); Tong Sun, Marlton, NJ (US); Shawn Watson, Cherry Hill, NJ (US); Kurt R. Nielsen, Chadds Ford, PA (US)

Correspondence Address: CHOATE, HALL & STEWART LLP, TWO INTERNATIONAL PLACE, BOSTON, MA, 02110, US

(73) Assignee: URL Pharma, Inc., Philadelphia, PA (US)

(21) Appl. No.: 12/332,795

(22) Filed: Dec. 11, 2008

Related U.S. Application Data
(60) Provisional application No. 61/015,959, filed on Dec. 21, 2007.

(51) Int. Cl. A61K 31/421; C07D 263/24; A61P 25/00

(52) U.S. Cl. 514/376; 548/232

(57) ABSTRACT

The present invention provides various amorphous forms of the compound metaxalone (I), such as solid amorphous metaxalone and amorphous dispersions comprising metaxalone. The present invention further provides pharmaceutical compositions comprising these amorphous forms, and methods of their preparation. The present invention additionally provides methods of treating painful conditions (e.g., such as painful musculoskeletal conditions) comprising administering a therapeutically effective amount of any one of these amorphous forms to a subject in need thereof.

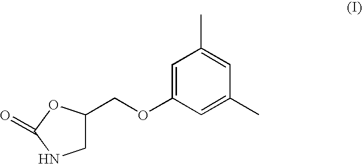

### RELATED APPLICATION

This application claims priority to provisional patent application U.S. Ser. No. 61/015,959, filed Dec. 21, 2007, the entire contents of which are hereby incorporated by reference.

### BACKGROUND OF THE INVENTION

The compound 5-(3′,5′-dimethylphenoxy)methyl-2-oxazolidinone of the structure (I):

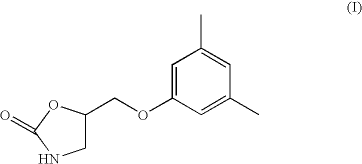

having the generic name Metaxalone, and marketed under the brand name SKELAXIN® (King Pharmaceuticals), has been described in several patents and applications for patent, such as U.S. Pat. Nos. 3,062,827; 3,446,814; 6,407,128; 6,538,142; 6,562,980; 6,683,102; and 7,122,566; United States Patent Application Publication Nos. 20050025717; 20050063913; 20050075505; 20050163839; 20050276844; and 20060167069; and International Publication Nos. WO 2006/082597, WO 2007/010508; WO 2007/074477; and WO 2007/079198; the entirety of each of which is hereby incorporated herein by reference. SKELAXIN® is formulated as a scoured tablet containing 800 mg of crystalline metaxalone.

Metaxalone, as an interneuronal blocking agent, acts on the central nervous systems (CNS) to produce muscle relaxant effects, and is used as an adjunct to rest, physical therapy, and other measures for the relief of discomforts associated with painful musculoskeletal conditions, such as pain and discomfort caused by muscle spasms, strains, sprains, tears and other muscle injuries. New therapeutic applications for metaxalone continue to surface, such as, for example, the treatment of diabetic neuropathy and chronic daily headache (Pfeifer et al., Diabetes Care (1993) 16:1103-1115; Ward, Postgrad Med. (2000) 108:121-128). The mode of action of metaxalone has not been clearly identified, but may be related to its sedative properties, and to general central nervous system depression. It is non-narcotic and non-addicting, with no adverse cardiovascular effects or interactions with MAOIs.

The pharmacokinetics of SKELAXIN® have been evaluated in healthy adult volunteers. Peak plasma concentrations of metaxalone occurred approximately 3 hours after a 400 mg oral dose under fasted conditions, with a mean Cₘₐₓ of 983 ng/mL, a mean Tₘₐₓ of 3.3 hours, a mean AUC of 7479 ng.h/mL and a mean t_{1/2} of 9.0 hours (SKELAXIN® medical insert). In a randomized, two way, crossover study, one 400 mg SKELAXIN® tablet was administered to healthy adult volunteers under fasted conditions, followed by a standard high-fat breakfast. Compared to fasted conditions, the high-fat meal at the time of drug administration increased the mean Cₘₐₓ by 177.5% and the mean AUC by 115.4%. Time-to-peak concentration (Tₘₐₓ) was also delayed by 1 hour, and terminal half life (t_{1/2}) was decreased by 6.6 hours under fed conditions compared to fasted. Other studies corroborate this food effect (see, for example, U.S. Pat. Nos. 6,407,128 and 6,683,102). Dissolution studies have attributed this food effect to the highly pH dependent dissolution of crystalline metaxalone from SKELAXIN® tablets. Crystalline metaxalone is insoluble at low pH (pH ˜1.5), and further, disintegration of the SKELAXIN® tablet at low pH is insufficient to enable metaxalone solubilization (Cacace et al., AAPS PharmSciTech (2004) 5:1-3). If the pH is raised to >3.0, after 1 hour, appreciable dissolution is achieved. Thus, if a patient takes a SKELAXIN® tablet on an empty stomach, it could be several hours before the product is exposed to a pH>3 that would effect its release.

There is therefore a need for new forms of metaxalone, especially new forms with improved physicochemical properties (e.g., solubility, stability, etc.) as compared to crystalline forms.

### SUMMARY OF THE INVENTION

The present invention provides various amorphous forms of the compound metaxalone (I), such as solid amorphous metaxalone and amorphous dispersions comprising metaxalone. The present invention further provides pharmaceutical compositions comprising these amorphous forms, and methods of their preparation. The present invention additionally provides methods of treating painful conditions (e.g., such as a painful musculoskeletal condition) comprising administering a therapeutically effective amount of any one of these amorphous forms to a subject in need thereof.

### BRIEF DESCRIPTION OF THE DRAWING

FIG. 1. XRPD patterns of metaxalone Forms A and B. Top: metaxalone Form A; Middle: metaxalone Form B; Bottom: post-moisture balance XRPD of metaxalone Form A

FIGS. 2A-2C. Comparison of FT-Raman spectra of metaxalone Forms A and B (Top: Form A, Bottom: Form B); FIG. 2A. Spectral range: 3600-100 cm⁻¹; FIG. 2B. Spectral range: 3260-2640 cm⁻¹; FIG. 2C. Spectral range: 1675-100 cm⁻¹.

FIG. 3. Thermal analysis of metaxalone Form A (Heating rate=10° C./min)

FIG. 4. Cyclic DSC thermogram of metaxalone Form A (Heating/cooling rate=30° C./min).

FIGS. 5A-5D. Hot-stage microscopy of metaxalone Form A; FIG. 5A. 28.2° C. (birefringence with extinction); FIG. 5B. 100.0° C. (birefringence with extinction); FIG. 5C. 121.5° C. (start of melt; melt complete by 122.6° C.); FIG. 5D. 167.5° C. (liquid on top slide boiling).

FIG. 6. Moisture sorption-desorption analysis of metaxalone Form A.

FIG. 7. Solid-state ¹³C-NMR spectrum of metaxalone Form A.

FIG. 8. Thermal analysis of metaxalone Form B (Heating rate=10° C./min).

FIG. 9. DSC thermogram of metaxalone Form B (Heating rate=10° C./min).

FIG. 10. Moisture sorption-desorption analysis of metaxalone Form B.

FIG. 11. Solid State ¹³C-NMR spectrum of metaxalone Form B.

FIG. 12. FT-Raman spectrum of metaxalone Form B.

FIGS. 13A-13D. XRPD patterns of amorphous dispersions of metaxalone. FIG. 13A. metaxalone and HPMC; FIG. 13B. metaxalone and HPMC-phthalate; FIG. 13C. metaxalone and HPMC-phthalate; FIG. 13D. metaxalone and PVP.

FIGS. 14A-14B. XRPD patterns of amorphous excipients. FIG. 14A. HPMC; FIG. 14B. HPMC-phthalate; FIG. 14C. PVP.

FIG. 15. Modulated DSC thermogram of an amorphous dispersion of metaxalone with HPMC-phthalate [Metaxalone: HPMC-phthalate (20:80)].

FIG. 16. Modulated DSC thermogram of an amorphous dispersion of metaxalone with PVP [Metaxalone:PVP (30:70)].

FIGS. 17A-17B. Modulated DSC thermogram of an amorphous dispersion of metaxalone with HPMC-phthalate [Metaxalone: HPMC-phthalate (50:50)]; FIG. 17A. underlying heating rate=2° C./min; FIG. 17B. underlying heating rate=1° C./min.

FIG. 18. Modulated DSC thermogram of an amorphous dispersion of metaxalone with HPMC [Metaxalone:HPMC (50:50)].

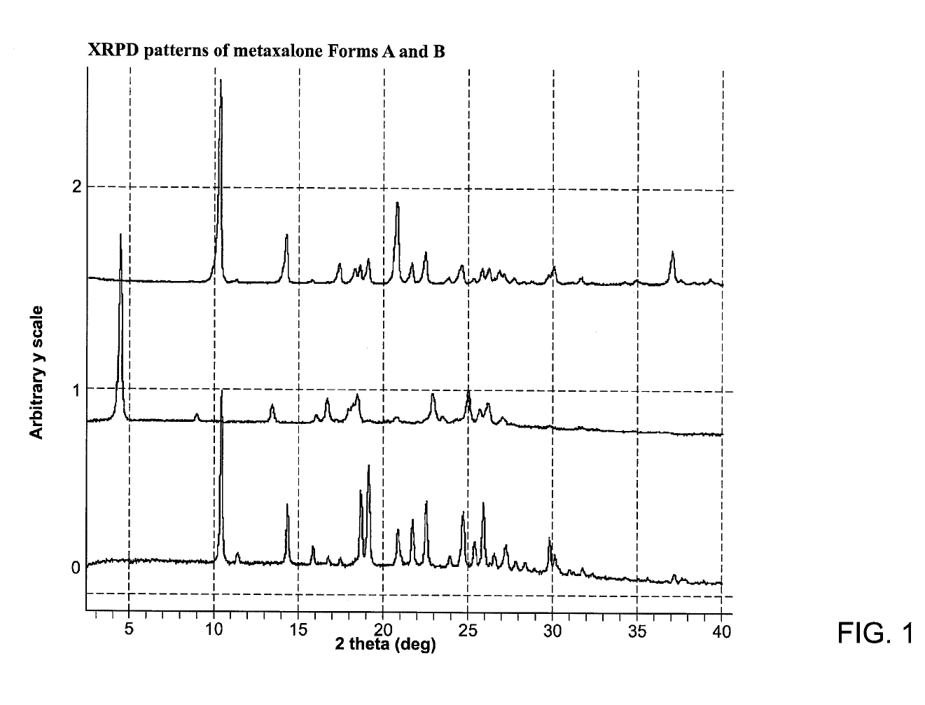

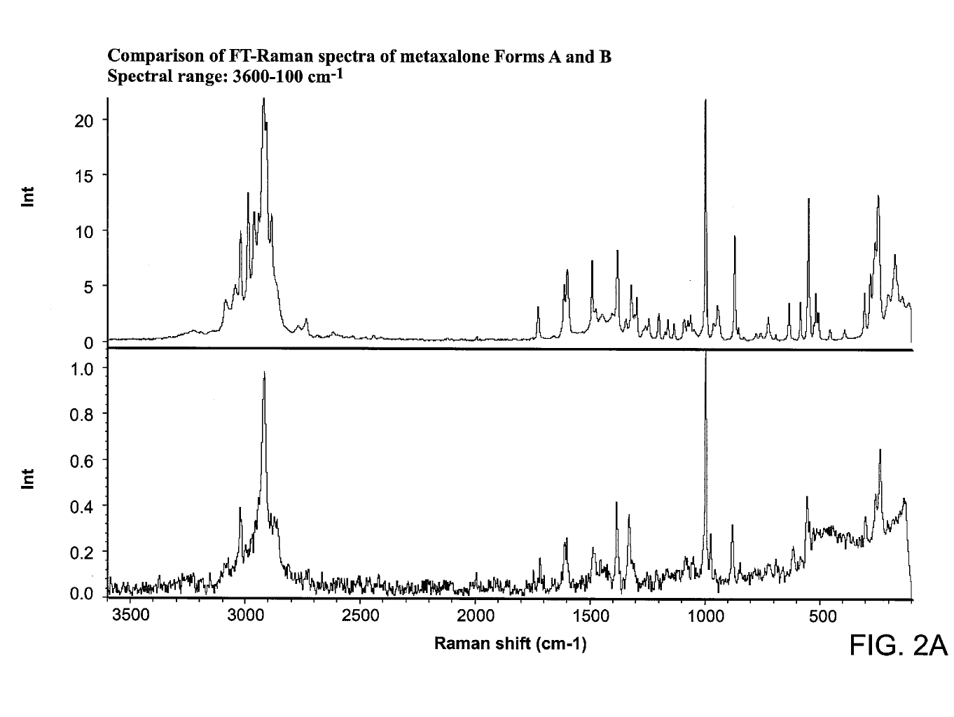

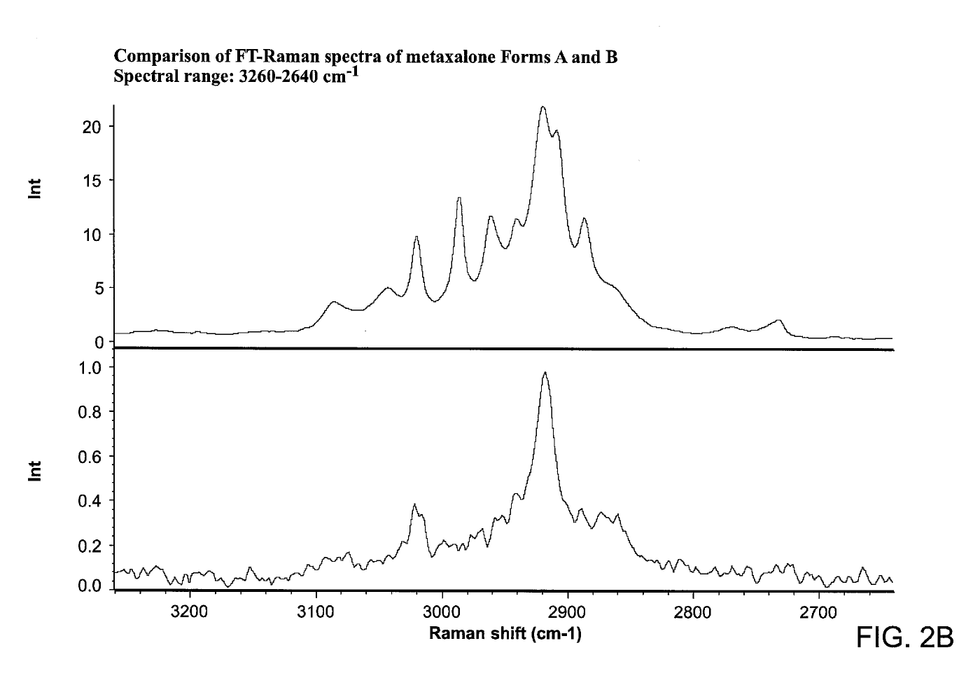

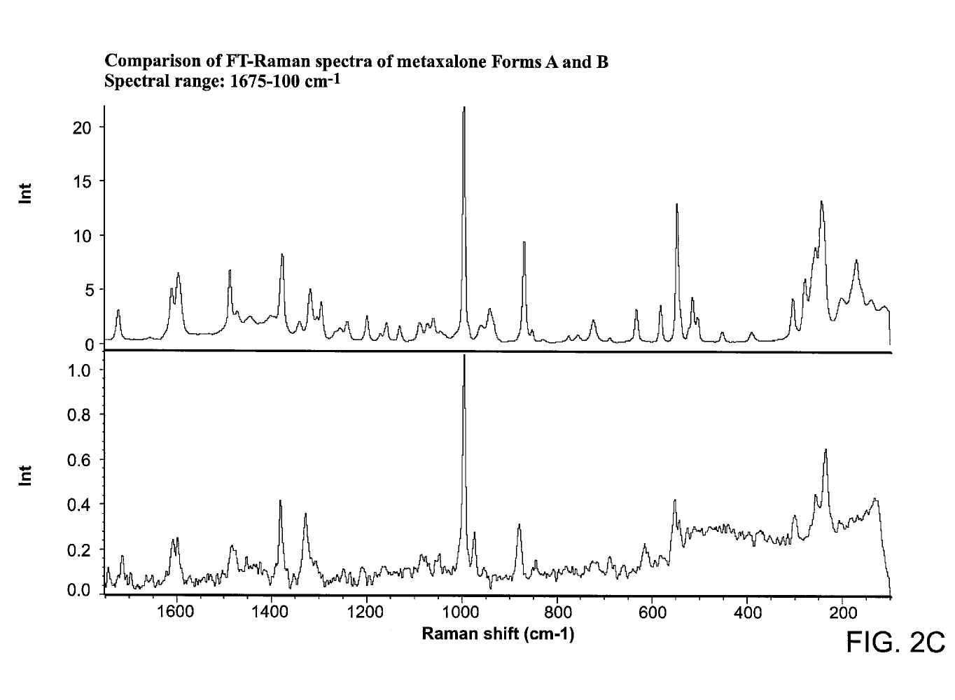

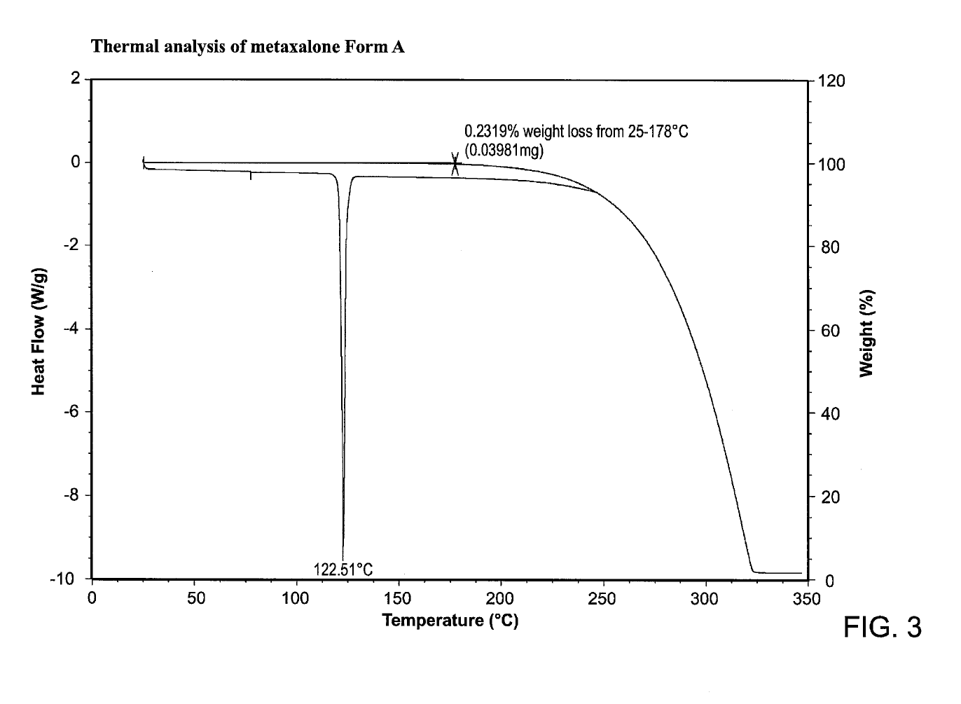

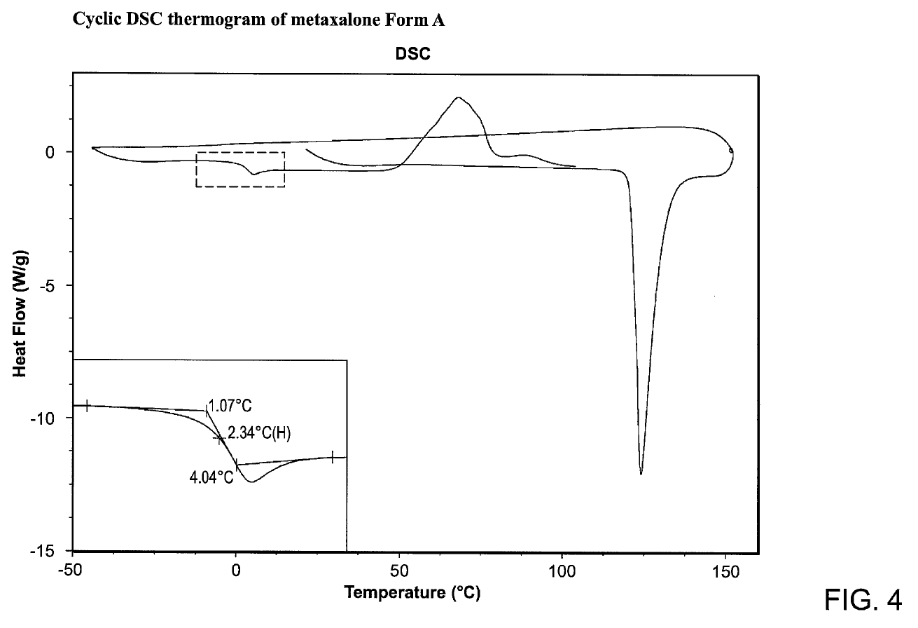

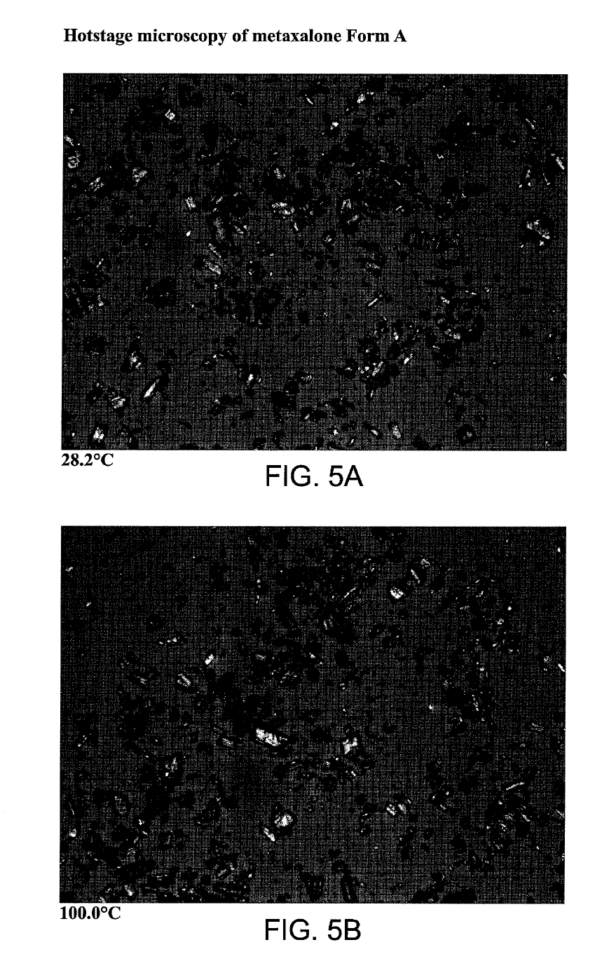

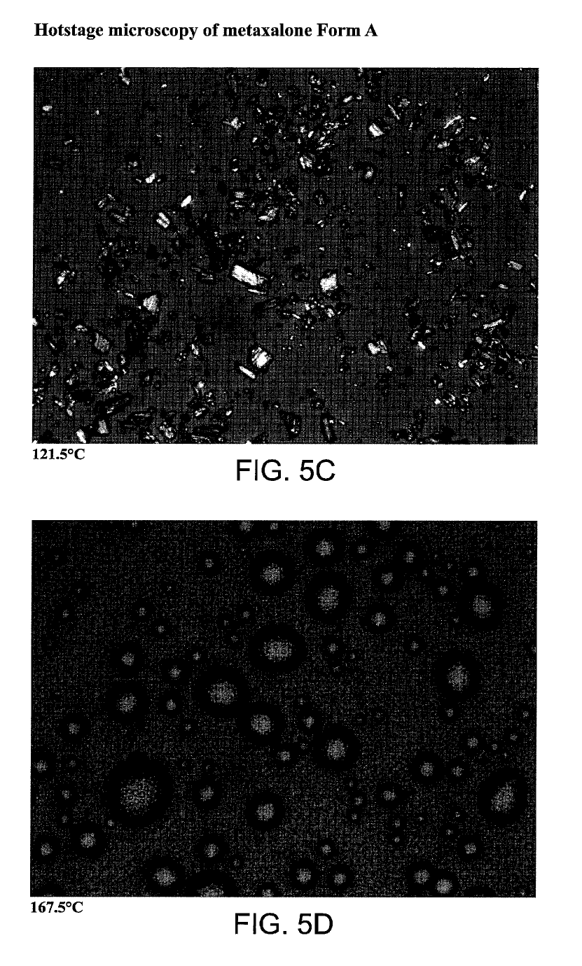

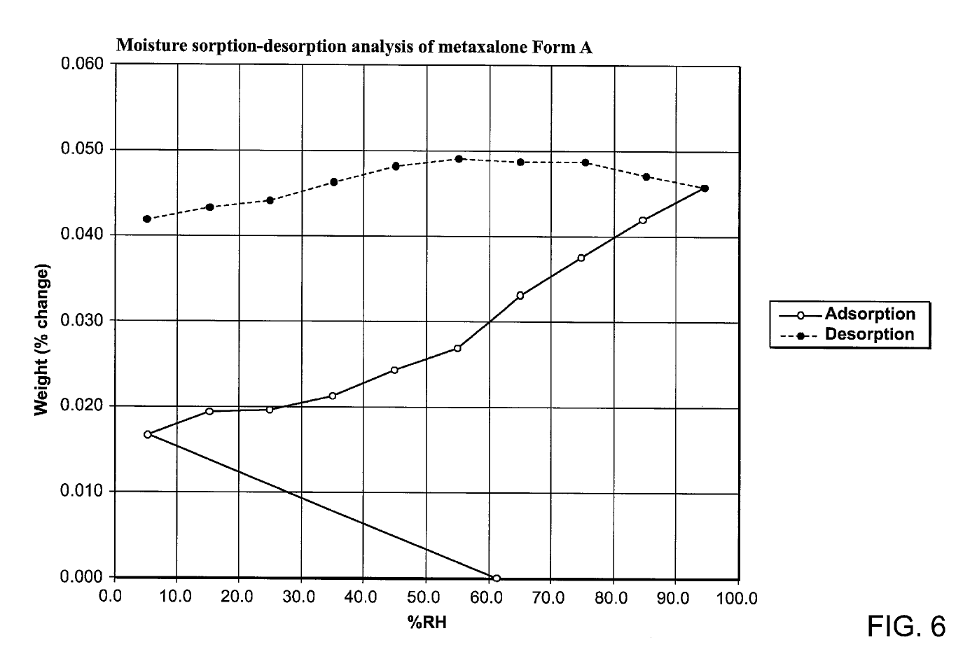

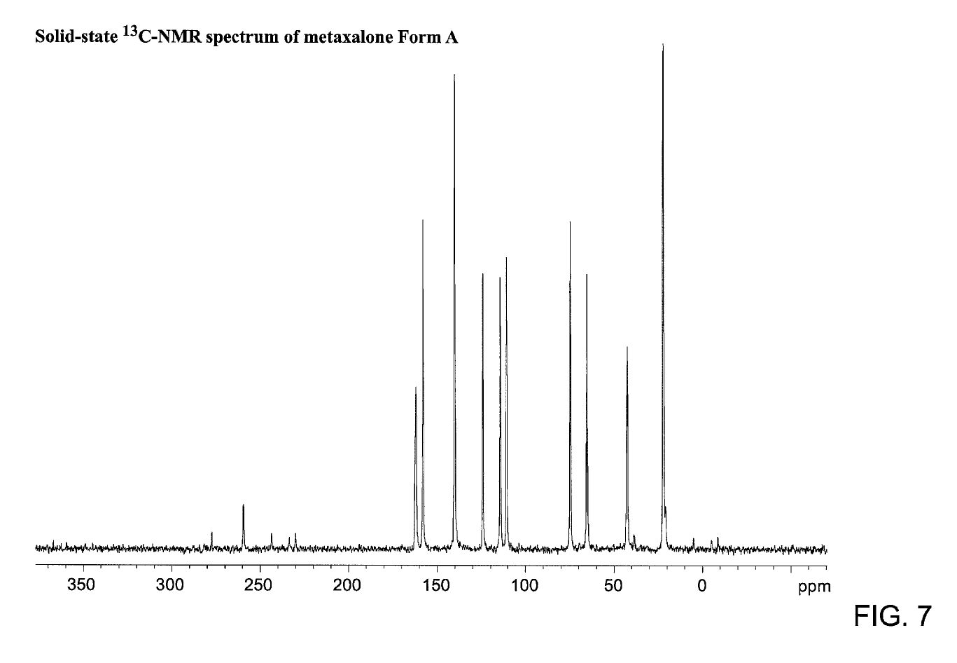

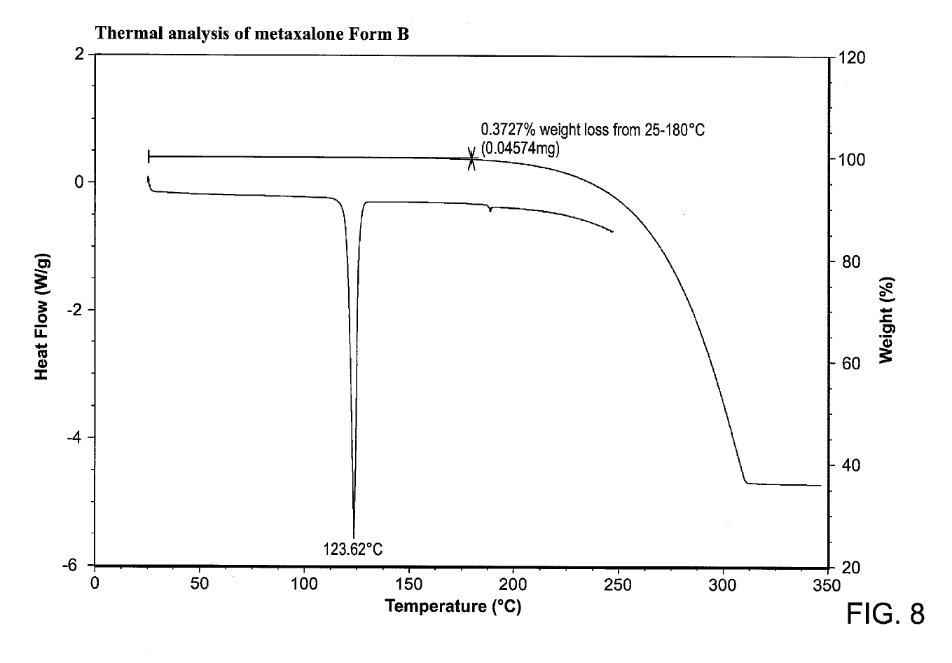

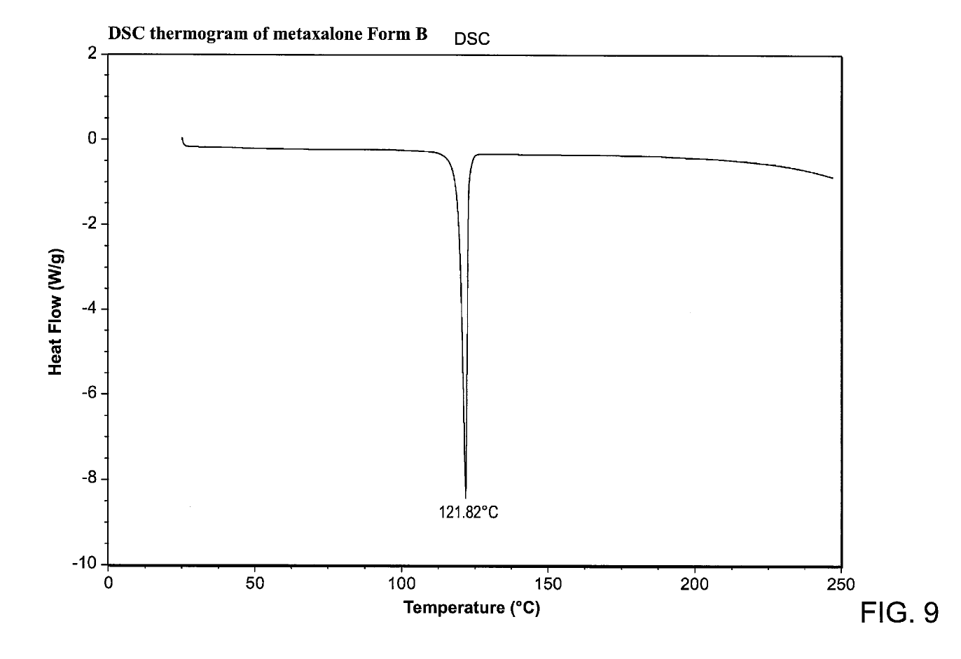

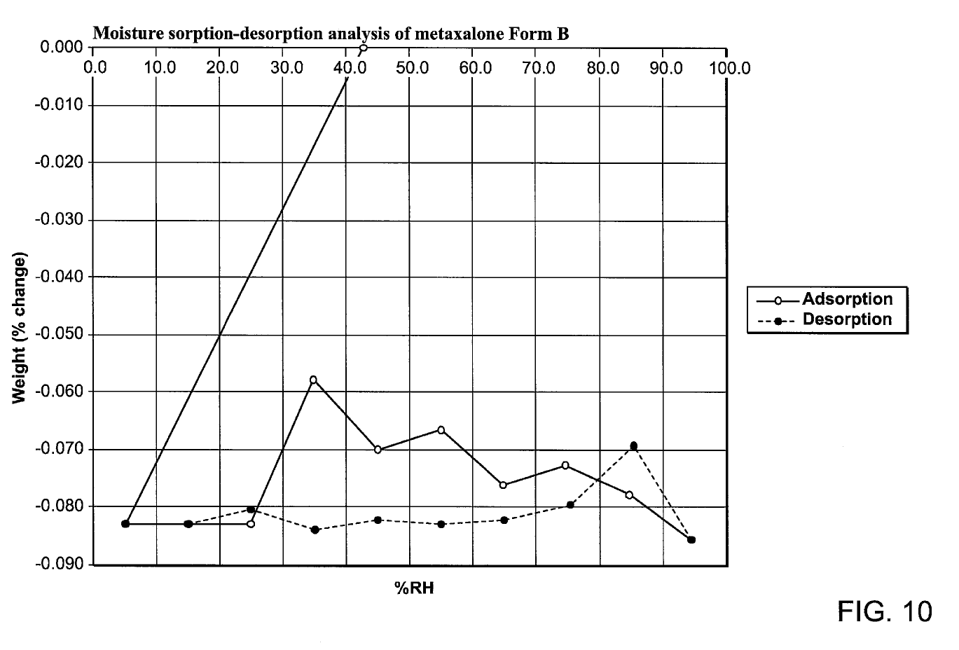

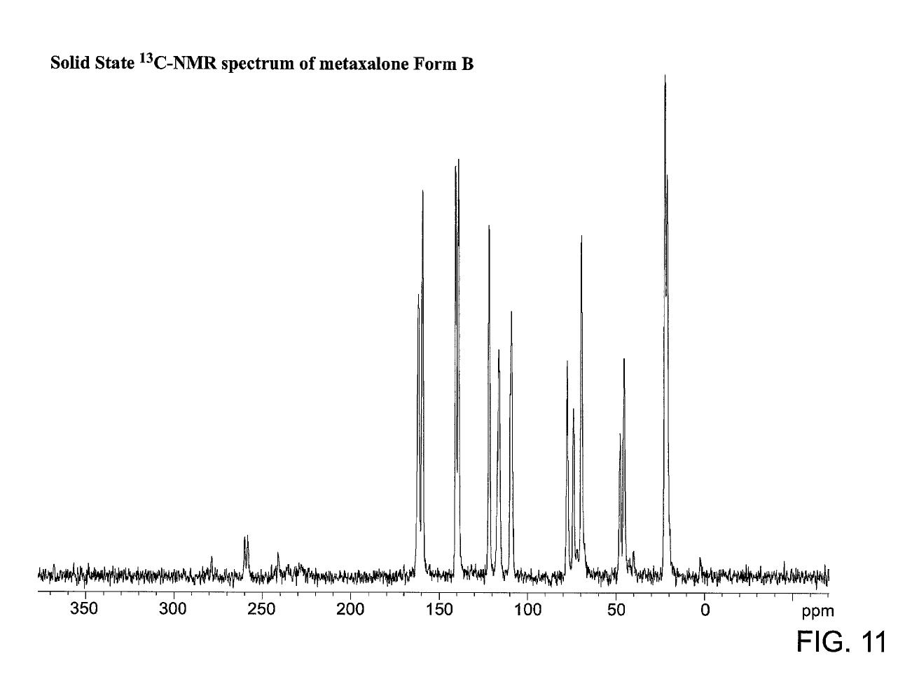

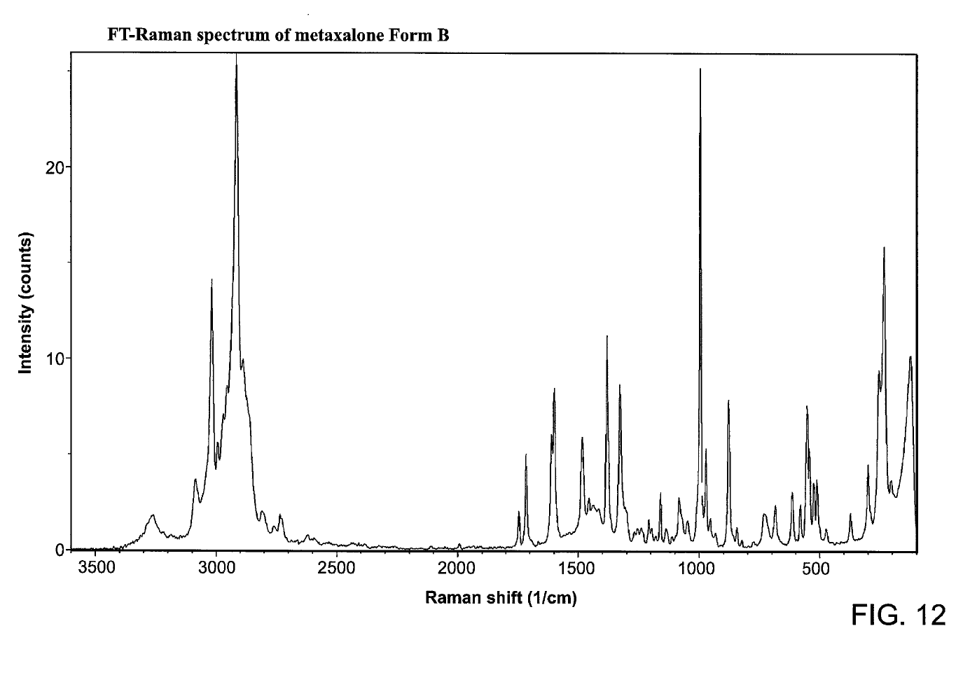

### DETAILED DESCRIPTION OF CERTAIN EMBODIMENTS OF THE INVENTION

While crystalline metaxalone, under the brand name SKELAXIN®, has been in the public domain for some time now, to our knowledge no method has been disclosed for generating a stable amorphous form of metaxalone. An amorphous form of metaxalone, such as an amorphous solid or an amorphous dispersion, particularly a stable form with improved physiochemical properties compared to crystalline metaxalone is therefore highly desirable.

### Amorphous Metaxalone

In one aspect, the present invention provides amorphous metaxalone. By “amorphous” is meant that metaxalone is not “crystalline.” By “crystalline” is meant that the compound metaxalone exhibits long-range order in three dimensions of at least 100 repeat units in each dimension. Thus, the term amorphous is intended to include not only material which has essentially no order, but also material which may have some small degree of order, but the order is in less than three dimensions and/or is only over short distances.

Amorphous material may be characterized by techniques known in the art such as Raman spectroscopy, IR spectroscopy (IR, FT-IR), powder X-ray diffraction (PXRD) crystallography, solid state NMR, microscopy (i.e., lack of birefringence), or thermal techniques such as differential scanning calorimetry (DSC) or Thermogravimetric analysis (TGA). Detectable amounts of crystalline metaxalone present in the amorphous material may be measured using these methods, or any other standard quantitative measurement.

The limits of detection of a particular form in admixture with another particular form, i.e. crystalline in amorphous, or vice versa, by XRPD is reported to be approximately 2% according to Surana and Suryanarayanan Powder Diffraction (2000) 15:2-6. The limits of detection by solution calorimetry is reported to be approximately 1% according to Hogan and Buckton, International Journal of Pharmaceutics (2000) 207:57-64. The limits of detection by solid state NMR is reported to be approximately 5 to 10% according to Saindonet al. Pharmaceutical Research (1993) 10:197-203. The limits of detection by near-IR spectroscopy is reported to be approximately 2 to 5% according to Blanco and Villar, Analyst (2000) 125:2311-2314. The limits of detection by modulated DSC is reported to be approximately 6% according to Saklatvala et al. International Journal of Pharmaceutics (1999) 192: 55-62. The limits of detection by Raman spectroscopy is reported to be approximately 2% according to Taylor and Zografi, Pharm. Res. (1998) 15:755-761, 1998).

Thus, in certain embodiments, solid amorphous metaxalone is substantially free of crystalline metaxalone. “Substantially free” in this context means that metaxalone is provided with less than about 10%, about 9%, about 8%, about 7%, about 6%, about 5%, about 4%, about 3%, about 2%, or less than about 1%, of crystalline metaxalone, e.g., crystalline Form A and/or crystalline Form B.

However, in other embodiments, solid amorphous metaxalone is provided as a mixture with crystalline metaxalone. Mixtures comprising crystalline metaxalone along with amorphous metaxalone may, depending on the amount of amorphous material present, possess varying levels of solubility. Such mixtures comprising amorphous metaxalone can be prepared, for example, by mixing amorphous metaxalone prepared according to the present invention with crystalline metaxalone. A mixture might also be prepared if the manufacturing process is incomplete, or incorporates steps that allow or causes both amorphous and/or crystalline material to be formed. A mixture might also be prepared if the solid amorphous form is unstable as we have found and converts partially to form an amount of crystalline material.

Thus, in certain embodiments, the present invention provides solid amorphous metaxalone as a mixture with crystalline metaxalone in a ratio of about 1:1, 1:2, 1:3, 1:4, 1:5, 1:6, 1:7, 1:8, 1:9, or about 1:10 of amorphous metaxalone to crystalline metaxalone. In certain embodiments, the present invention provides solid amorphous metaxalone as a mixture with crystalline metaxalone in a ratio of about 1:1, 1:2, 1:3, 1:4, 1:5, 1:6, 1:7, 1:8, 1:9, or about 1:10 of crystalline metaxalone to amorphous metaxalone.

Solid state ¹³C NMR spectroscopy (¹³C ssNMR) is one way of differentiating crystalline and amorphous forms. The isotropic chemical shifts (peak positions) measured in a solid state NMR spectra are not only a function of the molecule's atomic connectivity, but also of molecular conformation and inter- and intra-molecular interactions. Thus, different peak positions may be observed for different physical (amorphous or crystalline) forms. Furthermore, for amorphous solids, the dispersion of environments often causes substantially broadened spectra (R. K. Harris, Nuclear Magnetic Resonance Spectroscopy, (1987) Longman p. 155).

Amorphous solids and amorphous dispersions do not exhibit the three-dimensional long-range order found in crystalline materials, and therefore do not give a definitive x-ray diffraction pattern. Thus, another method of differentiating amorphous metaxalone and amorphous dispersions of metaxalone from crystalline metaxalone is by X-ray powder diffraction (XRPD).

### Amorphous Dispersions of Metaxalone

While we were able to prepare solid amorphous metaxalone we found that it was not stable and converted to a crystalline form. Thus, in another aspect, the present invention provides an amorphous dispersion of metaxalone. As discussed in the Examples, we were able to generate amorphous dispersions of metaxalone that were more stable than solid amorphous metaxalone. As used herein, an “amorphous dispersion” of metaxalone may be a solid dispersion (e.g., a wax, a polymeric matrix, a particle, a granule, a bead) or a liquid dispersion (e.g., an oil, a solution). Both solid and liquid amorphous dispersions comprise suspensions, partial suspensions, or homogenous dispersions of metaxalone in a dispersing aid. In certain embodiments, the present invention provides a solid or liquid amorphous dispersion of metaxalone as a suspension, a partial suspension, or homogenous dispersion of amorphous metaxalone substantially free of crystalline metaxalone in a dispersing aid. However, in certain embodiments, the present invention provides a solid or liquid amorphous dispersion of metaxalone as a suspension, a partial suspension, or homogenous dispersions of a mixture of amorphous and crystalline metaxalone in a dispersing aid.

As used herein a “dispersing aid” is a base which is used to suspend, or partially dissolve or partially suspend, or fully dissolve or homogenize metaxalone.

Exemplary dispersing aids include, but are not limited to, surface active agents and/or emulsifiers such as, for example, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), cellulosic derivatives (e.g., hydroxymethyl cellulose, hydroxypropyl cellulose, hydroxypropylmethylcellulose (HPMC), hydroxyethylcellulose, hydroxymethylcellulose, hydroxypropylmethylcellulose phthalate (HPMC-phthalate), methylcellulose), hydrated hydroxyalkylcellulose (e.g., OPADRY®), hydrated hydroxypropylmethylcellulose, carrageenan, alginates, sorbitan fatty acid esters (e.g., polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g., polyoxyethylene monostearate [Myrj 45], PEG-40 stearate [Myrij 52], polyoxyethylene hydrogenated castor oil, polyethoxylated castor oil, polyoxymethylene stearate, and Solutol), poly(vinylalcohols), sucrose fatty acid esters, polyethylene glycol (PEG, polyethylene glycol 8000), polyethylene glycol fatty acid esters (e.g., Cremophor), polyoxyethylene ethers, (e.g., polyoxyethylene lauryl ether [Brij 30]), poly(vinyl-pyrrolidone) (PVP), poly(vinyl-pyrrolidone)-vinyl acetate (PVP-VA), polymethacrylates (e.g., Acryl-EZE®, Acryl-EZE® MP, Surelease™, Eudragit® [e.g., Eudragit®L, Eudragit® RS-30D, Eudragit® RL-30D, Eudragit® L30-D55, Eudragit® L100, Eudragit® L100-55, Eudragit® S® 100, Eudragit® FS-30D]), diethylene glycol monolaurate, triethanolamine oleate, sodium oleate, potassium oleate, ethyl oleate, oleic acid, ethyl laurate, sodium lauryl sulfate, Pluronic F-68, Poloxamer 188, cetrimonium bromide, cetylpyridinium chloride, benzalkonium chloride, docusate sodium, etc. and/or combinations thereof.

Other exemplary dispersing aids include, but are not limited to, natural oils, such as, for example, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils.

Exemplary dispersing aids further include, but are not limited to, unnatural oils, such as, for example, butyl stearate, caprylic triglyceride, capric triglyceride, cyclomethicone, diethyl sebacate, dimethicone 360, isopropyl myristate, mineral oil, octyldodecanol, oleyl alcohol, and silicone oil.

In certain embodiments, the dispersing aid is selected from a group consisting of cellulosic derivatives, polyoxyethylene ethers or polymethacrylates. In certain embodiments, the cellulosic derivative is selected from a group consisting of hydroxypropylmethylcellulose (HPMC) and hydroxypropylmethylcellulose phthalate (HPMC-phthalate). In certain embodiments, the polyoxyethylene ether is selected from a group consisting of PEG-40 stearate [Myrij 52], poly(vinyl-pyrrolidone) (PVP) and poly(vinyl-pyrrolidone)-vinyl acetate (PVP-VA). In certain embodiments, the polymethacrylate is selected from a group consisting of Acryl-EZE®, Acryl-EZE® MP, Surelease™ or a Eudragit® (e.g., Eudragit® L, Eudragit® RS-30D, Eudragit® RL-30D, Eudragit® L30-D55, Eudragit® L100, Eudragit® L100-55, Eudragit® S 100, Eudragit® FS-30D).

The present invention also provides an amorphous dispersion of metaxalone characterized by an X-ray powder diffraction pattern lacking sharp diffraction peaks. In certain embodiments, the present invention provides an amorphous dispersion of metaxalone characterized by an X-ray powder diffraction pattern that contains one or more broad diffuse halos. In certain embodiments, the X-ray powder diffraction contains one broad diffuse halo. In certain embodiments, the X-ray powder diffraction contains two broad diffuse halos.

The term “broad diffuse halo” is the art recognized term for the “humps” observed in XRPD (Klug and Alexander, X-ray Diffraction Procedures: For Polycrystalline and Amorphous Materials, 2^{nd} Edition, John Wiley and Sons, New York, N.Y.: 1974, pp 791-792). It will be appreciated that a mixture comprising detectable amounts of both crystalline and amorphous metaxalone will exhibit both the characteristic sharp peaks and the broad diffuse halo(s) in an XRPD spectrum.

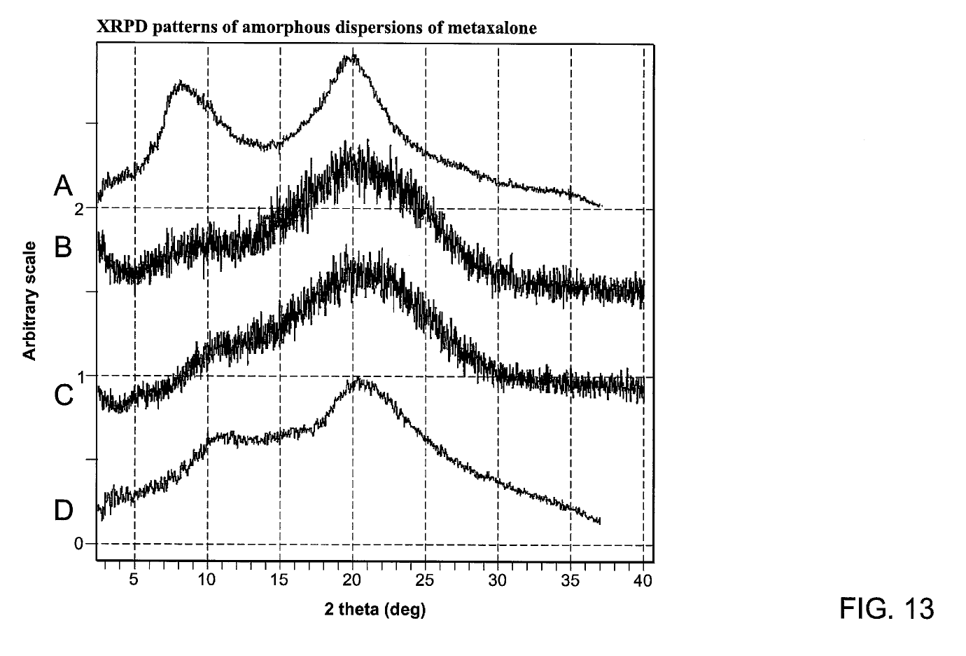

In certain embodiments, the dispersing aid is hydroxypropylmethylcellulose (HPMC). In certain embodiments, the dispersing aid is hydroxypropylmethylcellulose (HPMC) and the amorphous dispersion comprising metaxalone and HPMC is characterized by an X-ray powder diffraction pattern substantially similar to FIG. 13A. As shown in FIG. 13A, this dispersion has two characteristic broad diffuse halos. In one embodiment, these broad diffuse halos have maxima expressed in angle 2-theta at about 8 and 20 degrees.

In certain embodiments, the dispersing aid is hydroxypropylmethylcellulose phthalate (HPMC-phthalate). In certain embodiments, the dispersing aid is hydroxypropylmethylcellulose phthalate (HPMC-phthalate), and the amorphous dispersion comprising metaxalone and HPMC-phthalate is characterized by an X-ray powder diffraction pattern substantially similar to FIG. 13B and/or FIG. 13C. As shown in FIGS. 13B and C, this dispersion has at least one characteristic broad diffuse halo. In one embodiment, this broad diffuse halo has a maximum expressed in angle 2-theta at about 21 degrees. In some embodiments, this dispersion is prepared as a ˜20:80 combination of metaxalone to HPMC-phthalate and has a glass transition point (T_{g}), measured by DSC or TGA, of about 59° C. In other embodiments, this dispersion is prepared as a ˜50:50 combination of metaxalone to HPMC-phthalate and has a glass transition point (T_{g}), measured by DSC or TGA, of about 19° C.

In certain embodiments, the dispersing aid is poly(vinyl-pyrrolidone) (PVP). In certain embodiments, the dispersing aid is poly(vinyl-pyrrolidone) (PVP), and the amorphous dispersion comprising metaxalone and PVP is characterized by an X-ray powder diffraction pattern substantially similar to FIG. 13D. As shown in FIG. 13D, this dispersion has at least one characteristic broad diffuse halo. In one embodiment, this broad diffuse halo has a maximum expressed in angle 2-theta at about 21 degrees. In another embodiment, this dispersion has two broad diffuse halos with maxima expressed in angle 2-theta at about 12 and 21 degrees. In some embodiments, this dispersion has a glass transition point (T_{g}), measured by DSC or TGA, of about 75° C.

In certain embodiments, the dispersion includes at least about 5%, about 10%, about 20%, about 30%, about 40%, about 50%, about 60%, about 70%, about 80%, about 90%, or about 95% metaxalone by weight. In certain embodiments, the dispersion includes between about 10% to about 60% metaxalone by weight. In certain embodiments, the dispersion includes between about 15% to about 55% metaxalone by weight. In certain embodiments, the dispersion includes between about 20% to about 50% metaxalone by weight. In certain embodiments, the dispersion includes between about 20% to about 40% metaxalone by weight. In certain embodiments, the dispersion includes between about 30% to about 60% metaxalone by weight.

As discussed above for amorphous metaxalone, amorphous dispersions of metaxalone may, in certain embodiments, be substantially free of crystalline metaxalone. “Substantially free” in this context means that metaxalone in the amorphous dispersion, is provided with less than about 10%, about 9%, about 8%, about 7%, about 6%, about 5%, about 4%, about 3%, about 2%, or less than about 1%, of crystalline metaxalone, e.g., crystalline Form A and/or crystalline Form B. In other embodiments, an amorphous dispersion of metaxalone may comprise a mixture of amorphous metaxalone and crystalline metaxalone.

As discussed in the examples, we have found that different amorphous dispersions of amorphous metaxalone exhibit different levels of stability against conversion to crystalline metaxalone. “Stablility” refers to the tendency to remain substantially in the same physical (e.g., amorphous) state for a period of time (e.g., at least one week, at least one month, at least six months, at least a year, etc.). Stability can be assessed under different conditions, e.g., stressed conditions (60° C. at 75% relative humidity (RH)), ambient conditions (25° C. at 60% relative humidity (RH)) or under vacuum. Substantially the same physical state in this context means that at least about 70%, about 75%, about 80%, about 90%, about 95%, or about 99% of the amorphous metaxalone provided in the solid form or in a dispersion remains amorphous.

Thus, in certain embodiments, the present invention provides a stable amorphous dispersion of metaxalone. In certain embodiments, the present invention provides an amorphous dispersion of metaxalone which is stable for at least one week when stored under vacuum. In certain embodiments, the present invention provides an amorphous dispersion of metaxalone which is stable for at least two weeks when stored under vacuum. In certain embodiments, the present invention provides an amorphous dispersion of metaxalone which is stable for at least one month when stored under vacuum. In certain embodiments, the present invention provides a dispersion of amorphous metaxalone which is stable for at least two weeks when stored under ambient conditions. In certain embodiments, the present invention provides a dispersion of amorphous metaxalone which is stable for at least one week when stored under ambient conditions. In certain embodiments, the present invention provides a dispersion of amorphous metaxalone which is stable for at least one month when stored under ambient conditions.

### Methods of Preparing Amorphous Metaxalone

Still yet another aspect of the present invention is to provide methods for preparing amorphous metaxalone. In certain embodiments, the amorphous metaxalone is a glassy amorphous solid. In certain embodiments, the amorphous metaxalone is an amorphous powder.

In certain embodiments, the present invention provides a method for preparing amorphous metaxalone comprising the steps of (i) melting metaxalone and then (ii) cooling the molten product. In certain embodiments, amorphous metaxalone is prepared by initially melting crystalline metaxalone (e.g., crystalline Form A or B) at a temperature at or above its melting point (e.g., at or above about 121° C.). In certain embodiments, amorphous metaxalone is prepared by melting crystalline Form A. In certain embodiments, amorphous metaxalone is prepared by melting crystalline Form B.

In certain embodiments, the step of melting metaxalone comprises adding metaxalone to a container and heating the container. In certain embodiments, the container is open to the air. In certain embodiments, the container is sealed. In certain embodiments, the container is under inert atmosphere (e.g., such as a nitrogen or argon atomosphere).

In certain embodiments, the step of heating the container comprises heating to a temperature of between about 130° C. to about 200° C. In certain embodiments, the temperature is between about 140° C. to about 200° C. In certain embodiments, the temperature is between about 150° C. to 200° C. In certain embodiments, the temperature is between about 140° C. to 160° C. In certain embodiments, the temperature is about 155° C. In certain embodiments, the container is heated in an oil bath.

In other embodiments, the step of melting metaxalone comprises adding metaxalone to an approximately flat surface (e.g., a slide or plate), and heating the approximately flat surface. In certain embodiments, the metaxalone is melted in open air. In certain embodiments, the metaxalone is melted under a cover (e.g., a glass slide). In certain embodiments, the metaxalone is melted under inert atmosphere (e.g., such as a nitrogen or argon atmosphere).

In certain embodiments, the approximately flat surface is heated to a temperature of between about 130° C. to about 200° C. In certain embodiments, the approximately flat surface is heated to about 140° C. to about 200° C. In certain embodiments, the approximately flat surface is heated to between about 150° C. to about 200° C. In certain embodiments, the temperature is between about 140° C. to about 160° C. In certain embodiments, the approximately flat surface is heated to about 155° C.

In certain embodiments, the step of cooling comprises cooling to a temperature of between about 25° C. to about −100° C. In certain embodiments, the step of cooling comprises cooling to a temperature of between about 0° C. to about −100° C. In certain embodiments, the step of cooling comprises cooling to a temperature of between about −10° C. to about −100° C. In certain embodiments, the step of cooling comprises cooling to a temperature of between about −30° C. to about −100° C. In certain embodiments, the step of cooling comprises cooling to a temperature of between about −50° C. to about −100° C. In certain embodiments, the step of cooling comprises cooling to a temperature of between about −50° C. to about −80° C. In certain embodiments, the step of cooling comprises cooling to a temperature of about −78° C. (dry ice/acteone). In certain embodiments, cooling is immediate (i.e., by dropping down to the cooling temperature in a single step). In certain embodiments, cooling is gradual (e.g., by incremental cooling).

### Methods of Preparing Amorphous Dispersions of Metaxalone

In yet another aspect, the present invention provides methods for preparing amorphous dispersions of metaxalone. As generally described above, an “amorphous dispersion” of metaxalone may be a solid dispersion (e.g., a wax, a polymeric matrix, a particle, a granule, a bead) or a liquid dispersion (e.g., an oil, a solution). The amorphous dispersion may be phase separated in a suspension or partial suspension, meaning the compound metaxalone and the dispersing aid are each in separate domains within the amorphous dispersion, or the resulting amorphous dispersion may be homogeneous, meaning that the compound metaxalone and the dispersing aid are distributed throughout each other to form a single phase. In certain embodiments, the present invention provides a solid or liquid amorphous dispersion of metaxalone as a suspension, a partial suspension, or homogenous dispersion of amorphous metaxalone substantially free of crystalline metaxalone in a dispersing aid. However, in certain embodiments, the present invention provides a solid or liquid amorphous dispersion of metaxalone as a suspension, a partial suspension, or homogenous dispersions of a mixture of amorphous and crystalline metaxalone in a dispersing aid.

Preparation of such amorphous dispersions include, for example, mechanical, thermal and solvent processes. Exemplary mechanical processes include milling and extrusion; exemplary thermal processes including high temperature fusion, solvent-modified fusion and melt-congeal processes; and exemplary solvent processes including non-solvent precipitation, spray-coating and spray-drying. Often, these processes may form an amorphous dispersion by a combination of two or more process types. For example, when an extrusion process is used, the extruder may be operated at an elevated temperature such that both mechanical (shear) and thermal means (heat) are used to form the amorphous dispersion. Examples of methods used to form amorphous dispersions are disclosed in the following U.S. patents, the pertinent disclosures of which are incorporated herein by reference: U.S. Pat. Nos. 5,456,923 and 5,939,099, which describe forming dispersions by extrusion processes; U.S. Pat. Nos. 5,340,591 and 4,673,564, which describe forming dispersions by milling processes; and U.S. Pat. Nos. 5,707,646 and 4,894,235, which describe forming dispersions by melt congeal processes. Any of these preparative processes may be conducted in open air or under inert atmosphere (e.g., nitrogen, argon atmospheres).

Thus, in certain embodiments, the present invention provides a method of preparing an amorphous dispersion by a “mechanical process.” In certain embodiments, the present invention provides a method of preparing an amorphous dispersion comprising the step of milling metaxalone and a dispersing aid. In certain embodiments, the method further comprises the step of compacting metaxalone and a dispersing aid.

In other embodiments, the present invention provides a method of preparing an amorphous dispersion by a “thermal process.” In certain embodiments, the present invention provides a method of preparing an amorphous dispersion comprising the step of (i) melting together metaxalone and a dispersing aid, then (ii) cooling the molten product.

In certain embodiments, the dispersing aid is a solid at room temperature, and the step of melting comprises both melting the dispersing aid and melting metaxalone. However, in certain embodiments, the dispersing aid is a liquid at room temperature, and the step of melting comprises melting metaxalone into the liquid dispersing aid. In certain embodiments, an amorphous dispersion is prepared by melting crystalline metaxalone (e.g., crystalline Form A or B). In certain embodiments, an amorphous dispersion is prepared by melting crystalline Form A. In certain embodiments, an amorphous dispersion is prepared by melting crystalline Form B.

In certain embodiments, the step of melting comprises adding metaxalone and a dispersing aid to a container and heating the container. In certain embodiments, the container is open to the air. In certain embodiments, the container is sealed. In certain embodiments, the container is under inert atmosphere (e.g., such as a nitrogen or argon atomosphere).

In certain embodiments, the container is heated to a temperature of between about 100° C. to about 200° C. In certain embodiments, the temperature is between about 120° C. to about 200° C. In certain embodiments, the temperature is between about 130° C. to about 200° C. In certain embodiments, the temperature is between about 140° C. to about 200° C. In certain embodiments, the temperature is between about 120° C. to about 180° C. In certain embodiments, the temperature is between about 120° C. to about 160° C. In certain embodiments, the container is heated in an oil bath.

In other embodiments, the step of melting comprises adding metaxalone and a dispersing aid to an approximately flat surface (e.g., a slide, a plate), and heating the approximately flat surface. In certain embodiments, the metaxalone and dispersing aid are melted in open air. In certain embodiments, the metaxalone and dispersing aid are melted under a cover (e.g., a glass slide). In certain embodiments, the metaxalone and dispersing aid are melted under inert atmosphere (e.g., such as a nitrogen or argon atmosphere).

In certain embodiments, the approximately flat surface is heated to a temperature between about 100° C. to about 200° C. In certain embodiments, the temperature is between about 120° C. to about 200° C. In certain embodiments, the temperature is between about 130° C. to about 200° C. In certain embodiments, the temperature is between about 140° C. to about 200° C. In certain embodiments, the temperature is between about 120° C. to about 180° C. In certain embodiments, the temperature is between about 120° C. to about 160° C.

In certain embodiments, the step of cooling comprises cooling to a temperature of between about 25° C. to about −100° C. In certain embodiments, the step of cooling comprises cooling to a temperature of between about 0° C. to about −100° C. In certain embodiments, the step of cooling comprises cooling to a temperature of between about −10° C. to about −100° C. In certain embodiments, the step of cooling comprises cooling to a temperature of between about −30° C. to about −100° C. In certain embodiments, the step of cooling comprises cooling to a temperature of between about −50° C. to about −100° C. In certain embodiments, the step of cooling comprises cooling to a temperature of between about −50° C. to about −80° C. In certain embodiments, the step of cooling comprises cooling to a temperature of about −78° C. (dry ice/acteone). In certain embodiments, cooling is immediate (i.e., by dropping down to the cooling temperature in a single step). In certain embodiments, cooling is gradual (e.g., incremental cooling).

In yet other embodiments, the present invention provides a method of preparing an amorphous dispersion by a “solvent process.” In certain embodiments, the present invention provides a method of preparing an amorphous dispersion of metaxalone, comprising the steps of (i) dissolving at least a portion of metaxalone and at least a portion of a dispersing aid in a common solvent, and (ii) removing the common solvent.

A common solvent may be any solvent system (e.g., one solvent or a mixture of solvents) which in which metaxalone and the dispersing aid are soluble, or are at least partially soluble. In certain embodiments, the common solvent is a volatile solvent with a boiling point of 150° C. or less. Exemplary common solvents include organic alcohols such as methanol, ethanol, n-propanol, isopropyl alcohol (IPA), hexafluoroisopropyl alcohol (HFIPA), sec-butanol (methyl-1-propanol), and n-butanol; ketones such as acetone, methyl ethyl ketone, 3-pentanone, and methyl iso-butyl ketone; esters such as ethyl acetate and propylacetate; aromatic solvents such as toluene; chlorinated solvents such as dichloromethane (DCM), chloroform, and 1,1,1-trichloroethane, ethers such as tetrahydrofuran (THF), dioxane, diethyl ether, other solvents such as acetonitrile (ACN), and mixtures thereof.

The concentration of metaxalone and the dispersing aid in the common solvent depends on the solubility of each in the common solvent and the desired ratio of metaxalone to dispersing aid in the resulting amorphous dispersion. In certain embodiments, the common solvent comprises at least about 1 combined wt %, at least about 3 combined wt %, or at least about 10 combined wt % of metaxalone and dispersing aid.

In certain embodiments, the common solvent is present in the amorphous dispersion at a level that is acceptable according to The International Committee on Harmonization (ICH) guidelines. Reduction of the common solvent to this level may require additional drying steps, such as tray-drying, vacuum drying, fluid bed drying, microwave drying, belt drying, rotary drying, and other drying processes known in the art. To minimize chemical degradation, the additional drying step may take place under an inert gas such as nitrogen or argon, or may take place under vacuum.

In certain embodiments, the common solvent is rapidly removed (e.g., within 1 minute). In certain embodiments, the common solvent is slowly removed (e.g., greater than 1 minute). In certain embodiments, the step of removing the common solvent comprises removing by evaporation (e.g., rotary evaporation). In other embodiments, the step of removing the common solvent comprises removing by precipitation (e.g., by a change in temperature, pH, or addition of a solvent that induces precipitation). In yet other embodiments, the step of removing the common solvent comprises removing by spray-coating (e.g., pan-coating, fluidized bed coating, and the like). In still yet other embodiments, the common solvent is removed by spray-drying.

The term “spray-drying” is used conventionally and broadly refers to processes involving breaking up liquid mixtures into small droplets (atomization) and rapidly removing the common solvent from the mixture in a spray-drying apparatus where there is a strong driving force for evaporation of solvent from the droplets. Spray-drying processes and spray-drying equipment are described generally in Perry's Chemical Engineers Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, at least a portion of the heat required for evaporation of solvent may be provided by heating the spray solution.

The solvent-bearing feed can be spray-dried under a wide variety of conditions and yet still yield amorphous dispersions with acceptable properties. For example, various types of nozzles can be used to atomize the spray solution, thereby introducing the spray solution into the spray-dry chamber as a collection of small droplets. Essentially any type of nozzle may be used to spray the solution as long as the droplets that are formed are sufficiently small that they dry sufficiently (due to evaporation of the common solvent) that they do not stick to or coat the spray-drying chamber wall. Examples of types of nozzles that may be used to form the solid amorphous dispersions include the two-fluid nozzle, the fountain-type nozzle, the flat fan-type nozzle, the pressure nozzle and the rotary atomizer.

The maximum droplet size varies widely as a function of the size, shape and flow pattern within the spray-dryer. In certain embodiments, droplets are less than about 500 pm in diameter upon exiting the nozzle.

The spray solution can be delivered to the spray nozzle or nozzles at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas.

Generally, the energy for evaporation of solvent from the spray solution in a spray-drying process comes primarily from the drying gas. The drying gas can, in principle, be essentially any gas, but for safety reasons and to minimize undesirable decomposition of mexatalone or other materials in the solid amorphous dispersion, an inert gas such as nitrogen, nitrogen-enriched air or argon is utilized. The drying gas is typically introduced into the drying chamber at a temperature between about 60° and about 300° C. or between about 80° and about 240° C.

When the amorphous dispersion is formed using spray-drying techniques, the resulting dispersion are small solid particles. When the amorphous dispersion is formed by other methods, such by mechanical or thermal processes, the resulting amorphous dispersion may be sieved, ground, or otherwise processed to yield a plurality of small solid particles. The mean size of the particles may be less than about 500 um in diameter, less than about 200 um in diameter, less than about 100 um in diameter or less than about 50 um in diameter. In one embodiment, the particles have a mean diameter ranging from about 1 to about 100 um, or from about 1 to about 50 um.

For ease of processing, the dried particles may have certain density and size characteristics. In one embodiment, the resulting solid amorphous dispersion particles are formed by spray drying and may have a bulk specific volume of less than or equal to about 4 cc/g, or less than or equal to about 3.5 cc/g. In certain embodiments, the particles may have a tapped specific volume of less than or equal to about 3 cc/g, or less than or equal to about 2 cc/g. In certain embodiments, the particles have a Hausner ratio (ratio of the bulk specific volume to tapped specific volume) of less than or equal to about 3, or less than or equal to about 2. In certain embodiments, the particles have a Span of less than or equal to 3, or less than or equal to about 2.5. As used herein, “Span” is defined as:

$$Span = \frac{D [ {v}_{s} 0.9 ] - D [ {v}_{s} 0.1 ]}{D [ v , 0.5 ]}$$

wherein D[v,0.1] is the diameter corresponding to the diameter of particles that make up 10% of the total volume containing particles of equal or smaller diameter, D[v,0.5] is the diameter corresponding to the diameter of particles that make up 50% of the total volume containing particles of equal or smaller diameter, and D[v,0.9] is the diameter corresponding to the diameter of particles that make up 90% of the total volume containing particles of equal or smaller diameter.

Further descriptions of spray drying methods and other techniques for forming amorphous dispersions are provided in U.S. Pat. No. 6,763,607 and U.S. Patent Application No. 20060189633, the entirety of each of which is incorporated herein by reference.

### Pharmaceutical Compositions and Formulations

The present invention provides a pharmaceutical composition comprising amorphous metaxalone and one or more pharmaceutically acceptable excipients.

The present invention also provides a pharmaceutical composition comprising an amorphous dispersion of metaxalone and one or more pharmaceutically acceptable excipients.

For the purposes of the present invention, the phrase “active ingredient” generally refers to amorphous metaxalone, a mixture of amorphous metaxalone and crystalline metaxalone, or an amorphous dispersion comprising metaxalone, as described herein.

The formulations of the pharmaceutical compositions described herein may be prepared by any method known or hereafter developed in the art of pharmacology, such as those techniques described in M. E. Aulton in “Pharmaceutics: The Science of Dosage Form Design” (1988) (Churchill Livingstone) and Remington's Pharmaceutical Sciences, Sixteenth Edition, E. W. Martin (Mack Publishing Co., Easton, Pa., 1980), the entirety of each of which is incorporated herein by reference. In general, preparatory methods include the step of bringing the active ingredient into association with one or more pharmaceutically acceptable excipients and then, if necessary and/or desirable, shaping and/or packaging the product into a desired single- or multi-dose unit (e.g., a tablet, capsule, etc.).

Pharmaceutically acceptable excipients used in the manufacture of pharmaceutical compositions include, but are not limited to, solvent, inert diluents or other liquid vehicles, granulating and/or dispersing agents, surface active agents and/or emulsifiers, thickening agents, binding agents, preservatives, buffering agents, lubricating agents, and/or oils, as are described herein. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers/surfactants include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g., polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g., Cremophor), polyoxyethylene ethers, (e.g., polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, mannitol, etc.); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additional excipients such as glidants, lubricants, plasticizers, etc. may be included in the formulation. Glidants are agents used in solid dosage formulations to promote flowability of a solid mass. Such compounds include, by way of example and without limitation, colloidal silica, cornstarch, talc, calcium silicate, magnesium silicate, colloidal silicon, tribasic calcium phosphate, silicon hydrogel and other materials known to one of ordinary skill in the art. Lubricants, generally, are substances used in solid dosage formulations to reduce friction during compression. Such compounds include, by way of example and without limitation, sodium oleate, sodium stearate, calcium stearate, zinc stearate, magnesium stearate, polyethylene glycol, talc, mineral oil, stearic acid, sodium benzoate, sodium acetate, sodium chloride, and other materials known to one of ordinary skill in the art. Examples of suitable plasticizers include but are not limited to, dibutyl phthalate, diethyl phthalate, dibutyl sebacate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol, polyethylene glycol, propylene glycol and sorbitol.

Although the descriptions of the pharmaceutical compositions provided herein are principally directed to administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

The inventive metaxalone forms and pharmaceutical compositions thereof may be administered using any amount and any route of administration effective for treatment. Oral administration is the preferred route; however, the invention encompasses the delivery of the inventive metaxalone forms and pharmaceutical compositions thereof by any appropriate route taking into consideration likely advances in the sciences of drug delivery.

The specific therapeutically effective dose level for any particular subject or organism will depend upon a variety of factors including the condition being treated and the severity of the condition; the physiochemical behavior of the active ingredient; the specific composition employed; the age, body weight, general health, sex and diet of the subject; the time of administration, route of administration, and rate of excretion of the specific active ingredient and by-products; the duration of the treatment; drugs used in combination or coincidental with the active ingredient; and like factors well known in the medical arts.

The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

For example, the recommended dose for SKELAXIN® in adults and children over 12 years of age is one 800 mg tablet three to four times a day (SKELAXIN® medical insert). It will be appreciated that in certain embodiments, the inventive metaxalone forms may be combined with the excipients found in SKELAXIN® (i.e., alginic acid, ammonium calcium alginate, B-Rose Liquid, corn starch and magnesium stearate) to produce a pharmaceutical composition.

The inventive metaxalone forms and pharmaceutical compositions thereof are typically formulated in dosage unit form (e.g., 100, 200, 400 or 800 mg) for ease of administration and uniformity of dosage. It will be understood, however, that the total daily usage will be decided by the attending physician within the scope of sound medical judgment.

### Methods of Treatment and Use

It is still yet another aspect of the present invention to provide methods of treating a painful condition comprising administering a therapeutically effective amount of amorphous metaxalone to a subject in need thereof.

In yet another aspect, the present invention provides methods of treating a painful condition comprising administering a therapeutically effective amount of an amorphous dispersion of metaxalone to a subject in need thereof.

As used herein, a “painful condition” is meant a painful musculoskeletal condition, chronic or acute headache or diabetic neuropathy.

By “a painful musculoskeletal condition” is meant musculoskeletal complaints involving the muscles or components of the skeletal system. This includes the muscles themselves, the tendons and ligaments and other soft tissues such as the bursa (sacs of fluid that help in the lubrication of the joints). Exemplary musculoskeletal conditions include arthritis (e.g., osteoarthritis, inflammatory arthritis, rheumatoid arthritis, crystal arthritis), metabolic bone disease (e.g., osteoporosis), muscle spasms, and musculoskeletal injuries (e.g., sports-related injuries) such as back pain, foot pain, shoulder pain (e.g., tendinitis, frozen shoulder, rotator cuff syndrome), and muscle strains, tears and sprains.

In certain embodiments, the painful condition is a painful musculoskeletal condition. In certain embodiments, the painful musculoskeletal condition is muscle spasms and musculoskeletal injuries such as back pain, foot pain, shoulder pain, and muscle strains, tears and sprains. In certain embodiments, the painful musculoskeletal condition is acute.

Subjects to which administration is contemplated include, but are not limited to, humans (e.g., male, female, child, adolescent, adult, etc.) and other mammals, including primates and domesticated mammals such as cattle, pigs, horses, sheep, cats, and/or dogs.

“Treating,” as used herein, refers to partially or completely inhibiting, ameliorating, reducing, delaying, or diminishing the painful condition from which the subject is suffering. “Therapeutically effective amount,” as used herein, refers to the minimal amount or concentration of an inventive metaxalone form (e.g., amorphous metaxalone, mixture of amorphous metaxalone and crystalline metaxalone, or an amorphous dispersion, as are described above and herein), or pharmaceutical composition thereof, that, when administered, is sufficient in treating the subject.

A method for the treatment is provided comprising administering a therapeutically effective amount of an inventive metaxalone form or a pharmaceutical composition thereof to a subject in need thereof, in such amounts and for such time as is necessary to achieve the desired result. In certain embodiments of the present invention, a therapeutically effective amount of an inventive metaxalone form for administration one or more times a day to an adult human may comprise about 100 mg to about 1000 mg, about 200 to about 900 mg, about 400 to about 800 mg, about 400, or about 800 mg, of the inventive metaxalone form per unit dosage form. It will be appreciated that dose ranges as described herein provide guidance for the administration of inventive pharmaceutical compositions to an adult. The amount to be administered to, for example, a child or an adolescent can be determined by a medical practitioner or person skilled in the art and can be lower or the same as that administered to an adult.

It will be appreciated that an inventive metaxalone form or pharmaceutical composition thereof, as described above and herein, can be administered with food. Preferably the food is a solid food with sufficient bulk and fat content that it is not rapidly dissolved and absorbed in the stomach. In certain embodiments, the food is a meal, such as a breakfast, lunch or dinner. In some embodiments, the dosage is administered to the subject between about 30 minutes prior to about 2 hours after eating the meal. In certain embodiments, the dosage is administered to the subject within 15 minutes after eating a meal.

It will be also appreciated that an inventive metaxalone form or pharmaceutical composition thereof, as described above and herein, can be employed in combination with one or more additional therapeutically active agents. In general, each additional therapeutically active agent will be administered at a dose and/or on a time schedule determined for that agent. By “in combination with,” it is not intended to imply that the therapeutically active agents must be administered at the same time and/or formulated for delivery together, although these methods of delivery are within the scope of the invention. The compositions can be administered concurrently with, prior to, or subsequent to, one or more other additional therapeutically active agents. It will further be appreciated that the additional therapeutically active agent utilized in this combination may be administered together in a single composition or administered separately in different compositions. In general, it is expected that the additional therapeutically active agent be utilized at levels that do not exceed the levels at which they are utilized individually. In some embodiments, the levels utilized in combination will be lower than those utilized individually. The particular combination to employ in a regimen will take into account compatibility of the inventive metaxalone form with the additional therapeutically active agent and/or the desired therapeutic effect to be achieved.

The additional therapeutically active agent may achieve a desired effect for the condition being treated. For example, an inventive metaxalone form may be administered in combination with another muscle relaxant and/or a pain-relieving agent and/or an anti-inflammatory agent. Alternatively, the additional therapeutically active agent may achieve a different effect (e.g., by controlling an adverse effect, by improving the bioavailability, reducing and/or modifying the metabolism, inhibiting the excretion, and/or modifying the distribution of an inventive form of metaxalone within the body).

For example, in certain embodiments, an additional therapeutically active agent is a muscle relaxant. Exemplary muscle relaxants include carisoprodol (SOMA), cyclobenzaprine (FLEXERIL), methocarbamol (ROBAXIN), chlorzoxazone (PARAFON), baclofen (LIORESAL), dantrolene (DANTRIUM), orphenadrine (NORFLEX), tixanidine (ZANAFLEX), and diazepam (VALIUM).

In other embodiments, an additional therapeutically active agent is a pain relieving agent. Exemplary pain relieving agents include, but are not limited to, analgesics such as non-narcotic analgesics [e.g., salicylates such as aspirin, ibuprofen (MOTRIN®, ADVIL®), ketoprofen (ORUDIS®), naproxen (NAPROSYN®), acetaminophen, indomethacin] or narcotic analgesics [e.g., opioid analgesics such as tramadol, fentenyl, sufentanil, morphine, hydromorphone, codeine, oxycodone, and buprenorphine]; non-steroidal anti-inflammatory agents (NSAIDs) [e.g., aspirin, acetaminophen, COX-2 inhibitors]; steroids or anti-rheumatic agents; migraine preparations such as beta adrenergic blocking agents, ergot derivatives; tricyclic antidepressants (e.g., amitryptyline, desipramine, imipramine); anti-epileptics (e.g., clonaxepam, valproic acid, phenobarbital, phenyloin, tiagaine, gabapentin, carbamazepine, topiramate, sodium valproate); α₂ agonists; selective serotonin reuptake inhibitors (SSRIs), selective norepinepherine uptake inhibitors; benzodiazepines; mexiletine (MEXITIL); flecamide (TAMBOCOR); NMDA receptor antagonists [e.g., ketamine, detromethorphan, methadone]; and topical agents [e.g., capsaicin (Zostrix), EMLA cream, lidocaine, prilocaine].

In yet other embodiments, an additional therapeutically active agent is an anti-inflammatory agent. Exemplary anti-inflammatory agents include, but are not limited to, aspirin; ibuprofen; ketoprofen; naproxen; etodolac (LODINE®); COX-2 inhibitors such as celecoxib (CELEBREX®), rofecoxib (VIOXX®), valdecoxib (BEXTRA®), parecoxib, etoricoxib (MK663), deracoxib, 2-(4-ethoxy-phenyl)-3-(4-methanesulfonyl-phenyl)-pyrazolo[1,5-b]pyridazine, 4-(2-oxo-3-phenyl-2,3-dihydrooxazol-4-yl)benzenesulfonamide, darbufelone, flosulide, 4-(4-cyclohexyl-2-methyl-5-oxazolyl)-2-fluorobenzenesulfonamide), meloxicam, nimesulide, 1-Methylsulfonyl-4-(1,1-dimethyl-4-(4-fluorophenyl)cyclopenta-2,4-dien-3-yl)benzene, 4-(1,5-Dihydro-6-fluoro-7-methoxy-3-(trifluoromethyl)-(2)-benzothiopyrano(4,3-c)pyrazol-1-yl)benzenesulfonamide, 4,4-dimethyl-2-phenyl-3-(4-methylsulfonyl)phenyl)cyclo-butenone, 4-Amino-N-(4-(2-fluoro-5-trifluoromethyl)-thiazol-2-yl)-benzene sulfonamide, 1-(7-tert-butyl-2,3-dihydro-3,3-dimethyl-5-benzo-furanyl)-4-cyclopropyl butan-1-one, or their physiologically acceptable salts, esters or solvates; sulindac (CLINORIL®); diclofenac (VOLTAREN®); piroxicam (FELDENE®); diflunisal (DOLOBID®), nabumetone (RELAFEN®), oxaprozin (DAYPRO®), indomethacin (INDOCIN®); or steroids such as PEDIAPED® prednisolone sodium phosphate oral solution, SOLU-MEDROL® methylprednisolone sodium succinate for injection, PRELONE® brand prednisolone syrup.

Further examples of anti-inflammatory agents include naproxen, which is commercially available in the form of EC-NAPROSYN® delayed release tablets, NAPROSYN®, ANAPROX® and ANAPROX® DS tablets and NAPROSYN® suspension from Roche Labs, CELEBREX® brand of celecoxib tablets, VIOXX® brand of rofecoxib, CELESTONE® brand of betamethasone, CUPRAMINE® brand penicillamine capsules, DEPEN® brand titratable penicillamine tablets, DEPO-MEDROL brand of methylprednisolone acetate injectable suspension, ARAVA™ leflunomide tablets, AZULFIDIINE EN-tabs® brand of sulfasalazine delayed release tablets, FELDENE® brand piroxicam capsules, CATAFLAM® diclofenac potassium tablets, VOLTAREN® diclofenac sodium delayed release tablets, VOLTAREN®-XR diclofenac sodium extended release tablets, or ENBREL® etanerecept products.

One skilled in the art will recognize that some agents described herein act to relieve multiple conditions such as pain and inflammation, while other agents may just relieve one symptom such as pain. A specific example of an agent having multiple properties is aspirin, where aspirin is anti-inflammatory when given in high doses, but at lower doses is just an analgesic.

### EXAMPLES

In order that the invention described herein may be more fully understood, the following examples are set forth. It should be understood that these examples are for illustrative purposes only and are not to be construed as limiting this invention in any manner.

### Metaxalone Crystalline Form A

Metaxalone (21.5 mg) was added to a glass vial followed by acetonitrile (0.5 mL) and solids dissolved. The solution was filtered through 0.2 um nylon filter into a clean vial. The vial was uncapped and the solution was allowed to evaporate to dryness in a fumehood at ambient temperature. Solids were isolated after several days.

Metaxalone (22.8 mg) was added to a glass vial followed by ethyl acetate (1 mL) and solids dissolved. The solution was filtered through 0.2 um nylon filter into a clean vial. The vial was uncapped and the solution was allowed to evaporate to dryness in a fumehood at ambient temperature. Solids were isolated after several days.

Tables 1-4 provide data which characterize the crystalline form of metaxalone which was produced by these two methods (Form A). In various embodiments, the present disclosure provides metaxalone of Form A having one or more characteristic peaks from Table 3 and/or 4. In one embodiment, the present disclosure provides metaxalone of Form A having 2, 3, 4, 5, 6, 7, 8, 9 or 10 characteristic peaks from Table 3. In one embodiment, the present disclosure provides metaxalone of Form A having 2, 3, 4, 5, 6, 7, 8, 9 or 10 characteristic peaks from Table 4. In one embodiment, the present disclosure provides metaxalone of Form A having 2, 3, 4, 5, 6, 7, 8, 9 or 10 characteristic peaks from Table 3 and 2, 3, 4, 5, 6, 7, 8, 9 or 10 characteristic peaks from Table 4.

TABLE 1

| Conditions^{a} | Habit^{b} | Analysis^{c} | Result |
|---|---|---|---|
| EtOH/water SC, | B + E needles | SC-XRD | crystals too small |
| then slurry, | | | |
| then SC | | | |
| Sub-sample of above | B + E needles | XRPD | Form A |

^{a}SC = slow cool
^{b}B = birefringence, E = extinction. Observations made v polarized light microscopy.
^{c}SC-XRD = single crystal X-ray diffraction, XRPD = X-ray powder diffraction.

TABLE 2
Characterization of Metaxalone Form A

| Analysis^{a} | Result^{b} |
|---|---|
| XRPD | Form A |
| DSC | endotherm: 123° C. |
| TGA | negligible weight loss below 178° C. |
| cyclic-DSC | T_{g} = 2° C. (half height) |
| HSM | melt: 121° C.; liquid material starts boiling: 167° C. |
| MB | nonhygroscopic solid |
| ssNMR | unique |
| FT-Raman | unique |
| post-MB XRPD | Form A |

^{a}XRPD = X-ray powder diffraction, DSC = differential scanning calorimetry, TGA = thermogravimetric analysis, HSM = hotstage microscopy, MB = moisture balance, variable temperature X-ray powder diffraction ssNMR = solid state nuclear magnetic resonance spectroscopy, FT-Raman = Fourier Transform Raman spectroscopy.
^{b}Temperatures rounded to nearest degree.

TABLE 3
XRPD Peak Positions for Metaxalone Form A

| Peak No. | Peak position (°2θ) |
|---|---|
| 1 | 10.4 |
| 2 | 11.4 |
| 3 | 14.3 |
| 4 | 15.8 |
| 5 | 16.7 |
| 6 | 17.4 |
| 7 | 18.6 |
| 8 | 19.0 |
| 9 | 19.8 |
| 10 | 20.2 |
| 11 | 20.8 |
| 12 | 21.7 |
| 13 | 22.5 |
| 14 | 23.9 |
| 15 | 24.6 |
| 16 | 25.3 |
| 17 | 25.8 |
| 18 | 26.5 |
| 19 | 27.2 |
| 20 | 27.7 |
| 21 | 28.3 |
| 22 | 28.9 |
| 23 | 29.8 |
| 24 | 30.1 |
| 25 | 31.7 |
| 26 | 32.4 |
| 27 | 37.2 |
| 28 | 37.8 |

TABLE 4
Raman Peak Positions for Metaxalone Form A

| Peak No. | Peak position (cm⁻¹) | Peak No. | Peak position (cm⁻¹) |
|---|---|---|---|
| 1 | 110.5 | 1 | 1199.8 |
| 2 | 138.2 | 2 | 1241.5 |
| 3 | 169.1 | 3 | 1256.7 |
| 4 | 200.3 | 4 | 1295.8 |
| 5 | 242.3 | 5 | 1319.3 |
| 6 | 255.1 | 6 | 1342.0 |
| 7 | 277.1 | 7 | 1378.1 |
| 8 | 302.9 | 8 | 1446.5 |
| 9 | 390.0 | 9 | 1488.9 |
| 10 | 451.9 | 10 | 1597.0 |
| 11 | 503.6 | 11 | 1610.9 |
| 12 | 514.3 | 12 | 1723.5 |
| 13 | 546.6 | 13 | 1991.6 |
| 14 | 581.5 | 14 | 2442.5 |
| 15 | 632.5 | 15 | 2616.3 |
| 16 | 688.7 | 16 | 2732.3 |
| 17 | 723.1 | 17 | 2769.0 |
| 18 | 755.5 | 18 | 2886.5 |
| 19 | 775.2 | 19 | 2908.6 |
| 20 | 851.8 | 20 | 2919.3 |
| 21 | 868.8 | 21 | 2940.3 |
| 22 | 942 | 22 | 2961.1 |
| 23 | 995.9 | 23 | 2986.2 |
| 24 | 1060.5 | 24 | 3020.1 |
| 25 | 1072.4 | 25 | 3042.9 |
| 26 | 1089.0 | 26 | 3085.7 |
| 27 | 1131.2 | 27 | 3227.6 |
| 28 | 1158.8 | — | — |

### Metaxalone Crystalline Form B

Metaxalone (27.2 mg) was added to a glass vial followed by dichloromethane (0.5 mL) and solids dissolved. The solution was filtered through 0.2 um nylon filter into a clean vial. The vial was uncapped and the solution was allowed to evaporate to dryness in a fumehood at ambient temperature. Solids were isolated after several days.

Metaxalone (29.3 mg) was added to a glass vial followed by acetone (0.5 mL) and solids dissolved. The solution was filtered through 0.2 um nylon filter into a vial containing water (10 mL). Solids precipitated and were isolated by filtration.

Tables 5-8 provide data which characterize the crystalline form of metaxalone which was produced by these two methods (Form B). In various embodiments, the present disclosure provides metaxalone of Form A having one or more characteristic peaks from Table 7 and/or 8. In one embodiment, the present disclosure provides metaxalone of Form B having 2, 3, 4, 5, 6, 7, 8, 9 or 10 characteristic peaks from Table 7. In one embodiment, the present disclosure provides metaxalone of Form B having 2, 3, 4, 5, 6, 7, 8, 9 or 10 characteristic peaks from Table 8. In one embodiment, the present disclosure provides metaxalone of Form B having 2, 3, 4, 5, 6, 7, 8, 9 or 10 characteristic peaks from Table 7 and 2, 3, 4, 5, 6, 7, 8, 9 or 10 characteristic peaks from Table 8.

TABLE 5

| Conditions^{a} | Habit | Analysis^{b} | Result |
|---|---|---|---|
| ACN/US to SC | needles | SC-XRD | Form B |
| | | XRPD | Form B |

^{a}SC = slow cool, US = ultrasonication.
^{b}SC-XRD = single crystal X-ray diffraction, XRPD = X-ray powder diffraction.

TABLE 6
Characterization of Metaxalone Form B

| Preparation Conditions^{a} | Analysis^{b} | Result |
|---|---|---|
| DCM, FE | XRPD | Form B |
| | DSC | endotherm: 124° C. |
| | TGA | negligible weight loss |
| | | below 180° C. |
| lyophilization from | DSC | endotherm: 122° C. |
| dioxane | FT-Raman | unique |
| | MB | non-hygroscopic |
| | post MB-XRPD | Form B |
| lyophilization from | ssNMR | unique |
| dioxane | | |

^{a}FE = fast evaporation.
^{b}DSC = differential scanning calorimetry, TGA = thermogravimetric analysis, MB = moisture balance, VT-XRPD = variable temperature X-ray powder diffraction, FT-Raman = Fourier Transform Raman spectroscopy, SSNMR = solid-state nuclear magnetic resonance spectroscopy.

TABLE 7
XRPD Peak Positions for Metaxalone Form B

| Peak No. | Peak position (°2θ) |
|---|---|
| 1 | 4.45 |
| 2 | 8.95 |
| 3 | 13.4 |
| 4 | 16.0 |
| 5 | 16.7 |
| 6 | 17.2 |
| 7 | 17.9 |
| 8 | 18.1 |
| 9 | 18.4 |
| 10 | 19.2 |
| 11 | 20.2 |
| 12 | 20.8 |
| 13 | 22.00 |
| 14 | 22.9 |
| 15 | 23.5 |
| 16 | 24.3 |
| 17 | 24.9 |
| 18 | 25.6 |
| 19 | 26.1 |
| 20 | 27.0 |
| 21 | 27.4 |
| 22 | 29.8 |
| 23 | 31.6 |
| 24 | 34.7 |
| 25 | 36.3 |
| 26 | 36.7 |
| 27 | 38.8 |
| 28 | 39.5 |

TABLE 8
Raman Peak Positions for Metaxalone Form B

| Peak No. | Peak position (cm⁻¹) | Peak No. | Peak position (cm⁻¹) |
|---|---|---|---|
| 1 | 130.5 | 1 | 1111.3 |
| 2 | 204.8 | 2 | 1161.3 |
| 3 | 233.9 | 3 | 1210.8 |
| 4 | 254.4 | 4 | 1249.3 |
| 5 | 299.6 | 5 | 1329.0 |
| 6 | 314.3 | 6 | 1381.9 |
| 7 | 323.9 | 7 | 1417.1 |
| 8 | 372.0 | 8 | 1424.6 |
| 9 | 395.8 | 9 | 1452.9 |
| 10 | 409.4 | 10 | 1484.9 |
| 11 | 439.5 | 11 | 1598.6 |
| 12 | 448.1 | 12 | 1608.5 |
| 13 | 457.5 | 13 | 1715.1 |
| 14 | 510.3 | 14 | 1744.5 |
| 15 | 524.7 | 15 | 1994.7 |
| 16 | 542.4 | 16 | 2664.9 |
| 17 | 551.5 | 17 | 2724.8 |
| 18 | 582.0 | 18 | 2734.1 |
| 19 | 615.1 | 19 | 2756.8 |
| 20 | 658.9 | 20 | 2769.0 |
| 21 | 689.4 | 21 | 2811.0 |
| 22 | 725.1 | 22 | 2819.7 |
| 23 | 759.7 | 23 | 2860.3 |
| 24 | 780.7 | 24 | 2873.3 |
| 25 | 807.1 | 25 | 2889.5 |
| 26 | 843.9 | 26 | 2917.8 |
| 27 | 879.0 | 27 | 2952.1 |
| 28 | 954.5 | 28 | 2968.4 |
| 29 | 973.8 | 29 | 2998.6 |
| 30 | 995.4 | 30 | 3021.7 |
| 31 | 1037.1 | 31 | 3074.7 |
| 32 | 1047.7 | 32 | 3152.8 |
| 33 | 1085.4 | 33 | — |

### Structure Determination of Metaxalone Form B.

Cooling a saturated acetonitrile solution of metaxalone yielded colorless needles and the structure was determined by single crystal X-ray diffraction. The triclinic cell parameters and calculated volume are: a=5.5612(4) Å, b=10.3517(7) Å, c=19.8262(15) Å, α=82.582(3)°, β8=88.772(3)°, γ=82.597(5)°, V=1122.35(14) Å³. For Z=4 and formula weight=221.26 g/mol the calculated density is 1.31 g/cm³.

The quality of the structure obtained was reasonable, as indicated by an R-value of 6.8%. Usually R-values in the range of 2 to 6% are quoted for the most reliably determined structures (J. Glusker, K. Trueblood, Crystal Structure Analysis: A Primer, 2^{nd} ed.; Oxford University press: New York, 1985; p. 87).

### Solid Amorphous Metaxalone

A glass vial containing metaxalone Form A was placed in a silicon oil bath at 155° C. Once the solids had melted, the vial was quickly cooled in a dry ice/acetone bath (about −78° C.). Glassy solids resulted. The glassy solid was found to produce crystalline material when stored.

### Dispersions of Amorphous Metaxalone

An amorphous dispersion screen of metaxalone was carried out using a selection of dispersion aids. Dispersions were prepared by melting both components together (melt-quench) or by rapid evaporation from solution if both components were soluble (rotary evaporation).

TABLE 9

| Dispersing aid^{a} | Solvent | Conditions^{b} | Habit^{c} | Result |
|---|---|---|---|---|
| Eudragit L100 (38:62) | DCM^{k} | — | insoluble | — |
| Eudragit L100 (32:68) | acetone | — | insoluble | — |
| Eudragit L100 (46:54) | HFIPA^{l} | RE | B irr. shapes, mixed | — |
| | | | w/ non-B irr shapes | |
| Eudragit L100 (~50:50) | — | melt-quench | non-B irr. shapes | Form B + |
| | | @150° C. | | amorphous |
| HPMC (19:81)ⁱ | acetone | — | insoluble | — |
| HPMC (21:79) | DCM | — | insoluble | — |
| HPMC (48:52) | HFIPA | RE | wax-like solids, B | — |
| | | | irr. shapes, non-B | |
| | | | irr. shapes | |
| HPMC (~50:50) | — | melt quench | powdered solids, no | amorphous |
| | | @140° C. | B observed | |
| | | 26 days under vac | small B + E irr. | — |
| | | | shapes, non-B irr. | |
| | | | shapes | |
| HPMC-phthalate^{j} | acetone | RE | wax-like material | amorphous |
| (20:80) | | 27 days under vac | wax-like material | — |
| | | | with B | |
| HPMC-phthalate | acetone | RE | non-B wax-like | amorphous |
| (~50:50) | | | material | |
| | | 22 days under vac | B irr. shapes in | amorphous |
| | | | small amts., non-B | |
| | | | irr. shapes | |
| HPMC-phthalate | DCM | — | insoluble | — |
| (30:70) | | | | |
| HPMC-phthalate | — | melt quench | glass-like solids no | highly disordered |
| (~50:50) | | @140° C. | B observed | |
| PEG (17:83)^{e} | acetone | — | insoluble | — |
| PEG (11:89) | DCM | RE | wax-like material | Form B + peaks |
| PEG (35:65) | HFIPA | RE | clear tacky film to | — |
| | | | B aciculars (after | |
| | | | 30 min) | |
| PEG (~50:50) | — | melt quench | wax like solids B | — |
| | | @140° C. | observed | |
| Myrj 52 (36:64)^{f} | acetone | RE | clear film | — |
| Myrj 52 (36:64) | DCM | RE | clear film | — |
| PVP (MW = 360,000)^{g} | acetone | — | insoluble | — |
| (45:65) | | | | |
| PVP (MW = 360,000) | DCM | RE | wax-like material | amorphous |
| (30:70) | | 26 days under vac | B irr. shapes, non- | — |
| | | | B irr. shapes | |
| PVP (MW = 360,000) | DCM | RE | clear glass | — |
| (56:44) | | | | |
| PVP (MW = 360,000) | — | melt quench | glass-like solids B | — |
| (~50:50) | | @140° C. | irr. shapes | |
| PVP (MW = 1,300,000) | acetone | — | insoluble | — |
| (23:77) | | | | |
| PVP (MW = 1,300,000) | — | melt quench | non-B glass like | highly disordered |
| (~50:50) | | @140° C. | solids | |
| PVP (MW= 1,300,000) | DCM | RE | wax-like material | Form B |
| (23:77) | | | | |
| PVP-VA (20:80)^{h} | acetone | RE | glass | — |
| PVP-VA (20:80) | DCM | RE | glass | — |
| PVP-VA (~50:50) | — | melt quench | wax-like solids B | highly disordered |
| | | @140° C. | observed in small | |
| | | | amts | |

^{a}Ratio of metaxalone to dispersing aid listed in parentheses.
^{b}RE = rotary evaporation, LN = liquid nitrogen.
^{c}B = birefringence, irr. = irregular. Observations made visually or by using polarized light microscopy.
^{d}Sample considered non-GMP.
^{e}PEG = polyethyleneglycol
^{f}Myrj 52 = polyoxyl 40 Stearate
^{g}PVP = polyvinylpyrrolidone
^{h}PVP-VA = polyvinylpyrrolidone-vinyl acetate
ⁱHPMC = hydroxypropylmethylcellulose
^{j}HPMC-phthalate = hydroxypropylmethylcellulose phthalate
^{k}DCM = dichloromethane
^{l}HFIPA = hexafluoroisopropyl alcohol

Preparation of Amorphous dispersions of Metaxalone and HPMC (˜1:1). Approximately equal amounts of metaxalone Form A and HPMC were added to a glass vial and the vial was placed in an oil-bath at 140° C. The sample melted and was quickly plunged into a dry ice/acetone bath. Powdered solids were collected.

Preparation of Amorphous dispersions of Metaxalone and HPMC-Phthalate (20:80). Metaxalone Form A (13.1 mg) and HPMC-phthalate (51.7 mg) were added to a glass vial, followed by acetone (3 mL). Solids dissolved and solvent was removed under reduced pressure. A wax-like material was collected.

Preparation of Amorphous dispersions of Metaxalone and HPMC-Phthalate (1:1). Metaxalone Form A (24.5 mg) and HPMC-phthalate (23.5 mg) were added to a glass vial, followed by acetone (3 mL). Solids dissolved and the solution was filtered through 0.2 um nylon filter into a clean vial. The solvent was removed under reduced pressure. A wax-like material was collected.

Preparation of Amorphous dispersions of Metaxalone and PVP (30:70). Metaxalone Form A (18.2 mg) and PVP (42.7 mg) were added to a glass vial, followed by dichloromethane (3 mL). Solids dissolved and solvent was removed under reduced pressure. A wax-like material was collected.

### X-Ray Powder Diffractions (XRPD) of Amorphous Dispersions of Metaxalone.

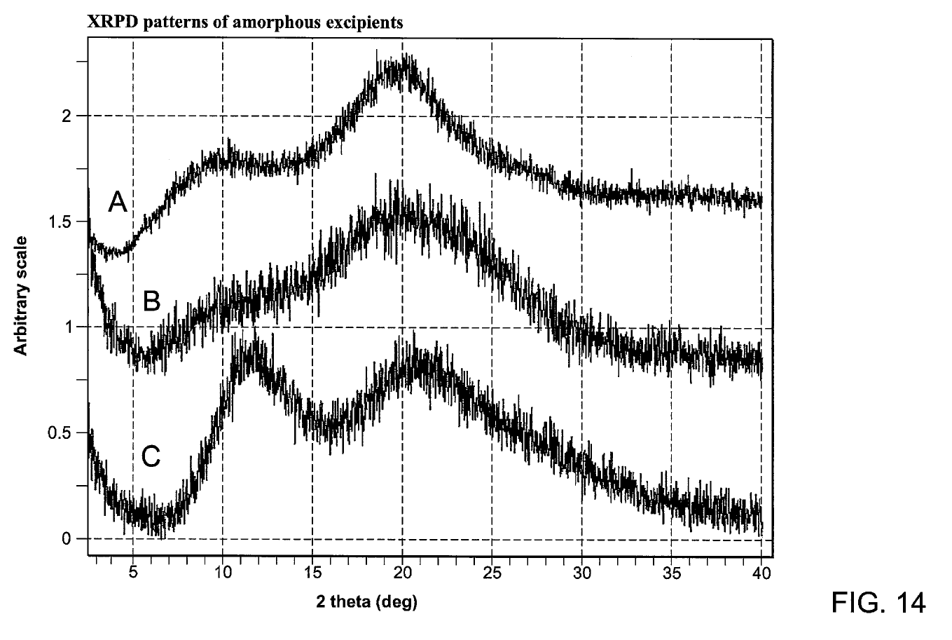

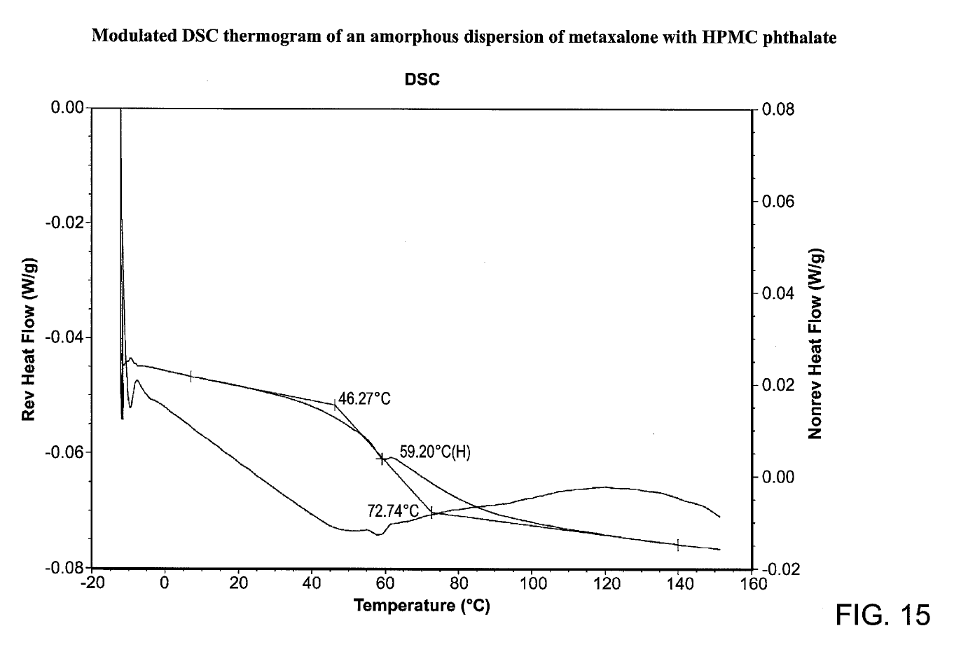

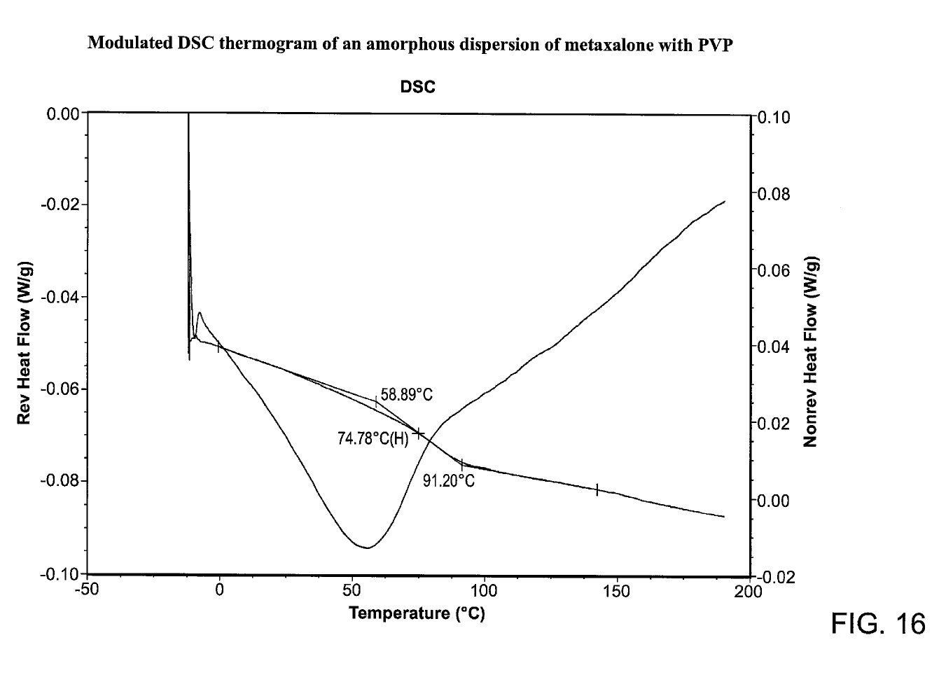

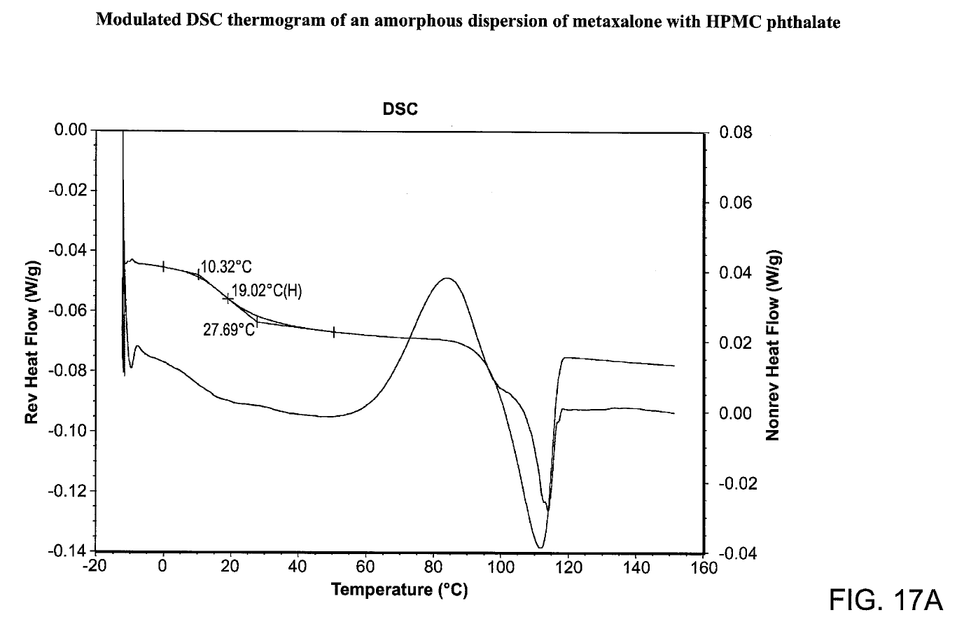

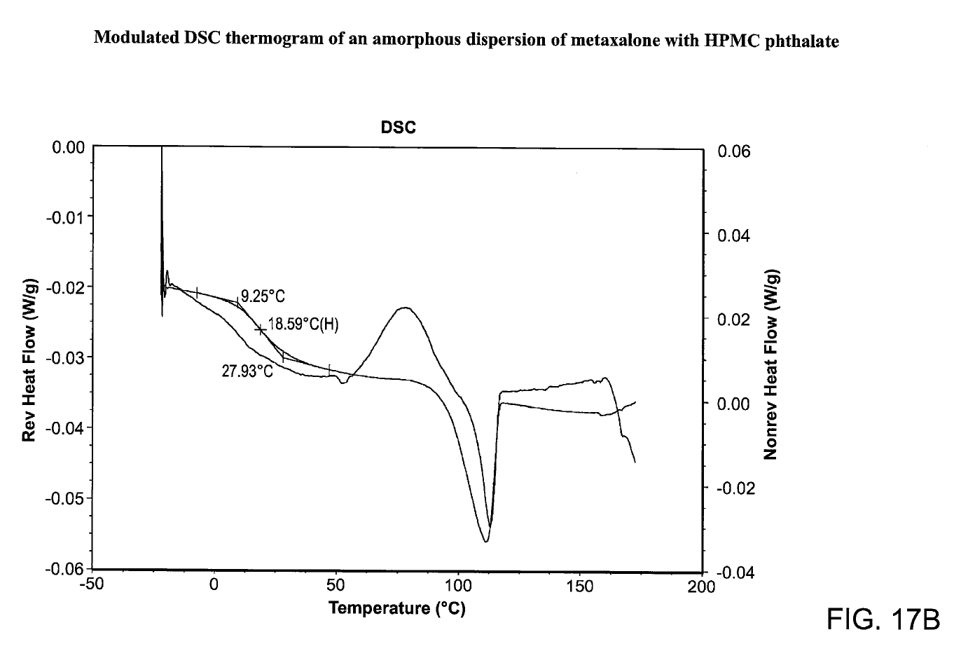

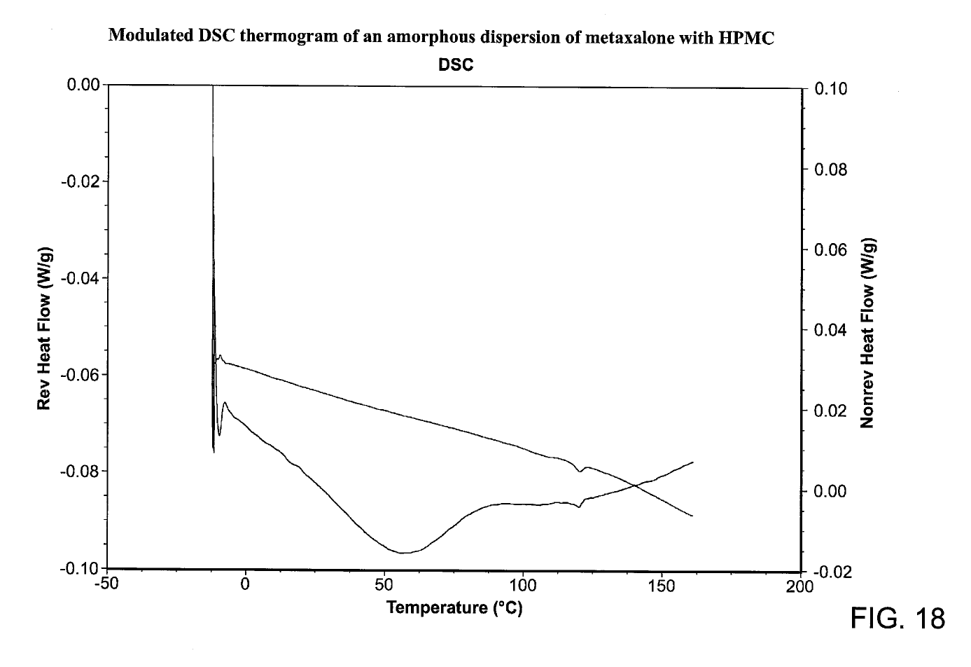

Most solids isolated exhibited crystallinity by XRPD but solids from HPMC (hydroxypropylmethylcellulose), HPMC-phthalate (hydroxypropylmethylcellulose phthalate) and PVP (polyvinylpyrrolidone) exhibited non-crystalline patterns (FIG. 13). XRPD patterns of the respective excipients (i.e., without metaxalone) are displayed in FIG. 14.

### Modulated DSC Analyses of Amorphous Dispersions of Metaxalone.

Glass transition temperatures were measured using modulated DSC (MDSC) but very broad inflexion points were noted in the thermograms and may represent glass transition temperatures (T_{g}) of non-ideal mixtures. The results are shown in Table 10.

TABLE 10

| Dispersion | Conditions^{a} | Result |
|---|---|---|
| HPMC (~50:50) | melt-quench @140° C. | — |
| HPMC-phthalate (20:80) | RE | T_{g} = 59° C.^{b} |
| HPMC-phthalate (~50:50) | RE | T_{g} = 19° C.^{b} |
| | >2 weeks under vacuum at RT | T_{g} = 19° C.^{c} |
| PVP (MW = 360,000) | RE | T_{g} = 75° C.^{b} |
| (30:70) | | |

^{a}RE = rotary evaporator, RT = room temperature.
^{b}Underlying heating rate = 2° C./min
^{c}Underlying heating rate = 1° C./min

### Stressing Studies of Amorphous Dispersions of Metaxalone.

Samples of non-crystalline dispersions were stressed under high relative humidity (RH) at elevated temperature for 1 week. All samples crystallized, indicating that the dispersions are not stable under high RH.

TABLE 11

| Dispersion | Conditions^{a} | Habit^{b} | Result |
|---|---|---|---|
| HPMC (~50:50) | ~75% RH, 60° C., 1 week | small B irr. shapes | Form B + HPMC |
| HPMC-phthalate | ~75% RH, 60° C., 1 week | non-B irr. shapes | Form B + HPMC |
| (20:80) | | | |
| HPMC-phthalate | ~75% RH, 60° C., 1 week | non-B irr. shapes, B irr. | Form B + amorphous |
| (~50:50) | | shapes in small amts | |
| Myrj 52 (36:64) | ~75% RH, 60° C., 1 week | B aciculars | Form A + peak |
| Myrj 52 (36:64) | ~75% RH, 60° C., 1 week | B aciculars | Form A + peak |
| PVP (MW = | ~75% RH, 60° C., 1 week | small B irr. shapes | Form B + PVP |
| 360,000) (30:70) | | | |
| PVP (MW = | ~75% RH, 60° C., 1 week | B acicular needles | Form B + amorphous |
| 360,000) (56:44) | | | |

^{a}RH = relative humidity.
^{b}B = birefringence, irr = irregular. Observations made using polarized light microscopy.

### Vacuum Studies of Amorphous Dispersions of Metaxalone.

The dispersions were also examined after approximately 3 weeks of storage under vacuum at ambient temperature. All samples exhibited evidence of crystallization except for metaxalone with HPMC-phthalate (˜50:50) which remained non-crystalline.

### Solubility Studies of Amorphous Dispersions of Metaxalone.

The dispersions are dissolved in different media that are commonly used in the art to simulate in vivo conditions (e.g., in the stomach, upper intestine, lower intestine, etc.) and dissolution profiles are obtained. These are then compared with dissolution profiles of crystalline material (e.g., Forms A or B) obtained under idential conditions. In one embodiment, the dissolution studies are performed using dispersions that have been stored under different conditions and for different periods of time to evaluate their long term stability.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. Amorphous metaxalone.

2. A pharmaceutical composition comprising amorphous metaxalone and one or more pharmaceutically acceptable excipients.

3. The pharmaceutical composition of claim 2, wherein said amorphous metaxalone is substantially free of crystalline metaxalone.

4. The pharmaceutical composition of claim 2, further comprising crystalline metaxalone.

5. A method of preparing amorphous metaxalone, comprising steps of (i) melting metaxalone to produce a molten product; and (ii) cooling the molten product to yield amorphous metaxalone.

6. The method according to claim 5, wherein the step of melting comprises heating the metaxalone to a temperature of between about 100° C. to 200° C.

7. The method according to claim 5, wherein the step of cooling comprises cooling the molten product to a temperature of between about 25° C. to −100° C.

8. The method according to claim 5, wherein the step of melting comprises heating the metaxalone to a temperature of between about 120° C. to 160° C. and the step of cooling comprises cooling the molten product to a temperature of between about −50° C. to −80° C.

9. The method according to claim 8, wherein the step of melting comprises melting crystalline metaxalone.

10. An amorphous dispersion comprising metaxalone and a dispersing aid.

11. The amorphous dispersion of claim 10, wherein the dispersing aid is HPMC and the amorphous dispersion exhibits an X-ray diffraction pattern with two broad diffuse halos having maxima expressed in angle 2-theta at about 8 degrees and about 20 degrees.

12. The amorphous dispersion of claim 10, wherein the dispersing aid is HPMC-phthalate and the amorphous dispersion exhibits an X-ray diffraction pattern with a broad diffuse halo having a maximum expressed in angle 2-theta at about 21 degrees.

13. The amorphous dispersion of claim 12, wherein metaxalone and the dispersing aid are present in about equal amounts by weight.

14. The amorphous dispersion of claim 10, wherein the dispersing aid is PVP and the amorphous dispersion exhibits an X-ray diffraction pattern with a broad diffuse halo having a maximum expressed in angle 2-theta at about 21 degrees.

15. The amorphous dispersion of claim 14, wherein the X-ray diffraction pattern includes a second weaker broad diffuse halo having a maximum expressed in angle 2-theta at about 12 degrees.

16. A pharmaceutical composition comprising an amorphous dispersion of claim 10 and one or more pharmaceutically acceptable excipients.

17. A method of preparing an amorphous dispersion of metaxalone comprising steps of (i) melting metaxalone and a dispersing aid to produce a molten product; and (ii) cooling the molten product to yield an amorphous dispersion.

18. The method according to claim 17, wherein the step of melting comprises heating the metaxalone and dispersing aid to a temperature of between about 100° C. to 200° C.

19. The method according to claim 17, wherein the step of cooling comprises cooling the molten product to a temperature of between about 25° C. to −100° C.

20. The method according to claim 17, wherein the step of melting comprises heating the metaxalone and dispersing aid to a temperature of between about 120° C. to 160° C. and the step of cooling comprises cooling the molten product to a temperature of between about −50° C. to −80° C.

21. A method of preparing an amorphous dispersion of metaxalone comprising steps of (i) dissolving at least a portion of metaxalone and at least a portion of a dispersing aid in a common solvent, and (ii) removing the common solvent to yield an amorphous dispersion.

22. The method according to claim 21, wherein the step of removing the common solvent comprises removal by evaporation or removal by spray-drying.

23. The method according to claim 22, wherein the common solvent is methanol, ethanol, n-propanol, isopropyl alcohol (IPA), hexafluoroisopropyl alcohol (HFIPA), sec-butanol, n-butanol, acetone, methyl ethyl ketone, 3-pentanone, methyl iso-butyl ketone, ethyl acetate, propylacetate, toluene, dichloromethane (DCM), chloroform, 1,1,1-trichloroethane, tetrahydrofuran (THF), dioxane, diethyl ether, acetonitrile (ACN), or a mixture thereof.

24. The method according to claim 22, wherein the common solvent is acetone or dichloromethane (DCM).

25. A method of treating a painful condition comprising administering a therapeutically effective amount of amorphous metaxalone to a patient in need thereof.

26. A method of treating a painful condition comprising administering a therapeutically effective amount of an amorphous dispersion of metaxalone comprising metaxalone and a dispersing aid to a patient in need thereof.
